(19) United States
(12) Patent Application Publication
(10) Pub. No.: US 2012/0040396 A1
(43) Pub. Date: Feb. 16, 2012

(54) METHODS FOR PURIFYING BIO-ORGANIC COMPOUNDS

(75) Inventors: Pinar Tabur, Emeryville, CA (US); Glenn Dorin, Emeryville, CA (US)

(73) Assignee: Amyris, Inc., Emeryville, CA (US)

(21) Appl. No.: 13/198,711

(22) Filed: Aug. 5, 2011

Related U.S. Application Data
(60) Provisional application No. 61/373,876, filed on Aug. 16, 2010.

(51) Int. Cl. C12P 39/00; C12P 5/02; C12N 1/15; C12N 1/19; C12N 1/13; C12P 5/00; C12N 1/21

(52) U.S. Cl. 435/42; 435/166; 435/167; 435/252.33; 435/252.31; 435/254.2; 435/254.21; 435/254.23; 435/257.2; 435/254.11

(57) ABSTRACT

Methods and systems for purifying bio-organic compounds are described. In certain embodiments, the methods comprise the steps of (a) providing a composition or an emulsion comprising a surfactant, host cells, an aqueous medium and a bio-organic compound produced by the host cells, wherein the solubility of the surfactant in the aqueous medium decreases with increasing temperature and wherein the temperature of the composition or emulsion is at least about 1° C. below a phase inversion temperature of the composition or emulsion; (b) raising the temperature of the composition or emulsion to at least about 1° C. above the phase inversion temperature; and (c) performing a liquid/liquid separation of the composition to provide a crude bio-organic composition or emulsion.

### PRIOR RELATED APPLICATION

This application claims the benefit under 35 U.S.C. §119(e) of U.S. Provisional Patent Application No. 61/373,876, filed Aug. 16, 2010, which is incorporated herein by reference in its entirety.

### FIELD OF THE INVENTION

Provided herein are methods for purifying microbial-derived bio-organic compounds. In some embodiments, the bio-organic compounds comprise one or more isoprenoids. In other embodiments, the bio-organic compounds comprise one or more farnesenes.

### BACKGROUND OF THE INVENTION

Petroleum-derived compounds and compositions are found in a variety of products ranging from plastics to household cleaners as well as fuels. Given the environmental impact of these compositions, there is an increasing demand for more renewable and sustainable alternatives.

Biological engineering can provide renewable sources for such compounds and compositions. For example, isoprenoids comprise a diverse class of compounds with over 50,000 members and have a variety of uses including as specialty chemicals, pharmaceuticals and fuels. Conventionally, isoprenoids can be synthesized from petroleum sources or extracted from plant sources. More recently, methods of making such compounds from microbial cells has been developed. For instance, isoprenoids and other microbial-derived compounds and compositions as well as methods of making them have been described in, for example, U.S. Pat. Nos. 7,399,323, 7,540,888, 7,671,245, 7,592,295, 7,589,243 and 7,655,739.

However, cost-effective methods of making and purifying such compounds are desired. For instance, methods for obtaining the optimal yields of a desired bio-organic compound are needed. Useful methods are provided herein.

### SUMMARY OF THE INVENTION

Provided herein are methods for purifying and/or isolating a microbial-derived bio-organic compound. In one aspect, provided herein is a method comprising:

(a) providing a composition comprising a surfactant, host cells, an aqueous medium and a bio-organic compound produced by the host cells, wherein the solubility of the surfactant in the aqueous medium decreases with increasing temperature and wherein the temperature of the composition is at least about 1° C. below a phase inversion temperature or a cloud point of the composition;

(b) raising the temperature of the composition to at least about 1° C. above the phase inversion temperature or the cloud point; and

(c) performing a liquid/liquid separation of the composition to provide a crude bio-organic composition.

In some embodiments, the method disclosed herein further comprising a step of reducing the volume of the composition before step (b) of raising the temperature of the composition, wherein substantially all of the bio-organic compound remains in the composition. In certain embodiments, the volume of the composition is reduced by about 75% or more. In some embodiments, the composition disclosed herein is an emulsion. In certain embodiments, the composition in step (a) above is an oil-in-water emulsion and the composition in steps (b) and (c) above is a water-in-oil emulsion.

In another aspect, provided herein is a method comprising:

(a) providing a first composition comprising a surfactant, host cells, an aqueous medium and a bio-organic compound produced by the host cells, wherein the solubility of the surfactant in the aqueous medium decreases with increasing temperature;

(b) concentrating the first composition to form a concentrated composition wherein the concentrated composition comprises substantially all of the bio-organic compound and the volume of the concentrated composition is less than the volume of the first composition, wherein the temperature of the concentrated composition is at least about 1° C. below a phase inversion temperature or a cloud point of the concentrated composition;

(c) raising the temperature of the concentrated composition to at least about 1° C. above the phase inversion temperature or the cloud point; and

(d) performing a liquid/liquid separation of the concentrated composition to provide a crude bio-organic composition.

In another aspect, provided herein is a composition comprising a surfactant, host cells, an aqueous medium and a bio-organic compound produced by the host cells, wherein the solubility of the surfactant in the aqueous medium decreases with increasing temperature and wherein the temperature of the composition is at least about 1° C. above a phase inversion temperature or a cloud point of the composition. In some embodiments, the composition is an emulsion. In certain embodiments, the composition is an oil-in-water emulsion. In other embodiments, the composition is a water-in-oil emulsion.

In another aspect, provided herein is an emulsion comprising a surfactant, host cells, an aqueous medium and a bio-organic compound produced by the host cells, wherein the solubility of the surfactant in the aqueous medium decreases with increasing temperature and wherein the temperature of the emulsion is at least about 1° C. above a phase inversion temperature or a cloud point of the emulsion.

In another aspect, provided herein is a method comprising:

(a) providing an oil-in-water emulsion comprising a surfactant, host cells, an aqueous medium and a bio-organic compound produced by the host cells, wherein the solubility of the surfactant in the aqueous medium decreases with increasing temperature;

(b) converting the oil-in-water emulsion to a water-in-oil emulsion; and

(c) performing a liquid/liquid separation of the water-in-oil emulsion to provide a crude bio-organic composition.

In some embodiments, the method disclosed herein further comprising a step of reducing the volume of the oil-in-water emulsion before step (b) of raising the temperature of the oil-in-water emulsion, wherein substantially all of the bio-organic compound remains in the composition. In certain embodiments, the volume of the oil-in-water emulsion is reduced by about 75% or more.

In another aspect, provided herein is a method comprising:

(a) providing a first oil-in-water emulsion comprising a surfactant, host cells, an aqueous medium and a bio-organic compound produced by the host cells, wherein the solubility of the surfactant in the aqueous medium decreases with increasing temperature;

(b) concentrating the first oil-in-water emulsion to form a concentrated oil-in-water emulsion wherein the concentrated oil-in-water emulsion comprises substantially all of the bio-organic compound and the volume of the concentrated oil-in-water emulsion is less than the volume of the first oil-in-water emulsion;

(c) converting the concentrated oil-in-water emulsion to a water-in-oil emulsion; and

(d) performing a liquid/liquid separation of the water-in-oil emulsion to provide a crude bio-organic composition.

### BRIEF DESCRIPTION OF DRAWINGS

FIG. 1 is a plot of oil recovery as a function of the concentration of surfactants including TERGITOL™ L62 and TERGITOL™ L64.

FIG. 2 is a plot of oil release rate as a function of the concentration of surfactants including TERGITOL™ L62 and TERGITOL™ L64.

FIG. 3 is a plot of oil recovery as a function of the concentration of surfactants including TERGITOL™ L62, TERGITOL™ L64, ECOSURF™ SA-7 and ECOSURF™ SA-9.

FIG. 4 is a plot of oil release rate as a function of the concentration of surfactants including TERGITOL™ L62, TERGITOL™ L64, ECOSURF™ SA-7 and ECOSURF™ SA-9.

FIG. 5 is a plot of oil release rate as a function of holding/mixing time with samples mixed with different methods including vortor mixer, rotating mixer, stir bar and ULTRA-TURRAX® disperser.

FIG. 6 is a plot of oil release rate as a function of mixing time by using ULTRA-TURRAX® disperser.

FIG. 7 is a plot of oil recovery as a function of concentration of TERGITOL™ L62. Two mixing methods including ULTRA-TURRAX® disperser and stir bar were investigated.

FIG. 8 is a plot of oil release rate as a function of concentration of TERGITOL™ L62. Two mixing methods including ULTRA-TURRAX® disperser and stir bar were investigated.

### DETAILED DESCRIPTION OF THE INVENTION

### Terminology

“Crude bio-organic composition” refers to a composition comprising a bio-organic compound wherein the bio-organic compound is present in an amount at least about 75% by weight of the crude bio-organic composition. In some embodiments, the bio-organic compound is present in an amount at most about 80%, about 85%, about 87% or about 89% by weight of the crude bio-organic composition.

“Bio-organic compound” refers to a water-immiscible compound that is made by microbial cells (both recombinant as well as naturally occurring). In certain embodiments, the bio-organic compound is a hydrocarbon. In certain embodiments, the bio-organic compound is a C₄-C₃₀ containing compound or hydrocarbon. In certain embodiments, the bio-organic compound is an isoprenoid. In certain embodiments, the bio-organic compound is a C₅-C₂₀ isoprenoid. In certain embodiments, the bio-organic compound is a C₁₀-C₁₅ isoprenoid.

“Phase inversion temperature” or “PIT” refers to the temperature at which the continuous and dispersed phases of an emulsion system are inverted (e.g., an oil-in-water emulsion becomes a water-in-oil emulsion, and vice versa).

“Cloud point” refers to the temperature at which one or more liquids and/or solids dissolved in a fluid are no longer completely soluble, precipitating as a second phase giving the fluid a cloudy appearance.

“Phenolic antioxidant” refers to an antioxidant that is a phenol or a phenol derivative, wherein the phenol derivative contains an unfused phenyl ring with one or more hydroxyl substituents. The term also includes polyphenols. Illustrative examples of a phenolic antioxidant include: resveratrol; 3-tert-butyl-4-hydroxyanisole; 2-tert-butyl-4-hydroxyanisole; 4-tert-butylcatechol (which is also known as TBC); 2,4-dimethyl-6-tert-butylphenol; and 2,6-di-tert-butyl-4-methylphenol (which is also known as butylhydroxytoluene or BHT). Additional examples of phenolic antioxidants are disclosed in U.S. Pat. No. 7,179,311.

“Purified bio-organic composition” refers to a composition comprising a bio-organic compound wherein the bio-organic compound is present in the composition in an amount equal to or greater than about 90% by weight. In certain embodiments, the bio-organic compound is present in an amount equal to or greater than about 95%, about 96%, about 97%, about 98%, about 99% or about 99.5% by weight.

“Polished composition” refers to a purified bio-organic composition that is further treated, for example, to reduce formation of peroxides in the composition or to stabilize the composition with an anti-oxidant or treated with a chelating agent to reduce the amounts of metals in the compositions.

“Process(es)” refers to a purification method(s) disclosed herein that is (are) useful for isolating a microbial-derived organic compound. Modifications to the methods disclosed herein (e.g., starting materials, reagents) are also encompassed.

In the following description, all numbers disclosed herein are approximate values, regardless whether the word “about” or “approximate” is used in connection therewith. Numbers may vary by 1 percent, 2 percent, 5 percent or, sometimes, 10 to 20 percent. Whenever a numerical range with a lower limit, R_{L}, and an upper limit, R_{U}, is disclosed, any number falling within the range is specifically disclosed. In particular, the following numbers within the range are specifically disclosed: R=R_{L}+k*(R_{U}−R_{L}), wherein k is a variable ranging from 1 percent to 100 percent with a 1 percent increment, i.e., k is 1 percent, 2 percent, 3 percent, 4 percent, 5 percent, . . . , 50 percent, 51 percent, 52 percent, . . . , 95 percent, 96 percent, 97 percent, 98 percent, 99 percent or 100 percent. Moreover, any numerical range defined by two R numbers as defined in the above is also specifically disclosed.

The claimed subject matter can be understood more fully by reference to the following detailed description and illustrative examples, which are intended to exemplify non-limiting embodiments.

Purification Methods

Provided herein are methods for purifying the bio-organic compounds disclosed herein. The bio-organic compounds can be made using any technique deemed suitable by one of skill in the art. Some non-limiting examples of bio-organic compounds include isoprenoids made using methods such as those described in U.S. Pat. Nos. 7,399,323 and 7,659,097; and PCT Publication Nos. WO 2007/140339, WO 2008/140492, WO 2008/133658 and WO 2009/014636, all of which are incorporated herein by reference in their entireties. Other examples include fatty-acid derived olefins such as those described in U.S. Patent Publication No. 2009/0047721; and PCT Publication Nos. WO 2008/113041 and WO 2008/151149, all of which are incorporated herein by reference in their entireties.

Although there are many publications describing microbial methods for producing bio-organic compounds, there are relatively few publications describing purification methods for such compounds from fermentation or other biological production systems. For example, PCT Publication WO 2007/139924 relates to systems for making bio-organic compounds and describes purification methods which generally rely on the inherent tendency for the bio-organic compound to separate from an aqueous medium. However, although this separation does occur and purified bio-organic compounds can be obtained, there can be significant product losses due to emulsion formation.

In general, an emulsion is a mixture of two immiscible liquids, such as water and an oil (e.g., a bio-organic compound). Mechanical energy from either fermentation (e.g. from agitators or fermentation gases produced by host cells) or downstream processing can promote emulsion formation where a bio-organic compound is produced and subsequently extracted into, for example, an aqueous fermentation medium. Moreover, as described by various literature references, host cells as well as various bio-molecules therein can also promote and/or stabilize emulsion formation. For the above reasons, emulsion formation is inevitable in a microbial production system. Therefore, a simple and scalable purification method that destabilizes an emulsion can be useful for purifying a microbial-derived bio-organic compound cost-effectively.

Provided herein are purification methods that reliably and consistently destabilize an emulsion and provide cost-effective purification methods for a microbial-derived bio-organic compound. In general, the method relies on first forming a chemically defined emulsion in an aqueous medium such as fermentation broth. The formation of this emulsion is mediated by the addition of a surfactant whose solubility in an aqueous medium decreases with increasing temperature and the temperature of the aqueous medium is below its phase inversion temperature or cloud point. The resulting emulsion is then destabilized by increasing the temperature of the composition to above its phase inversion temperature or cloud point. In certain embodiments, the emulsions that are first formed are oil-in-water emulsions. In some embodiments, the oil-in-water emulsions are destabilized to form the corresponding water-in-oil emulsions.

In one aspect, provided herein are methods that comprise:

(a) providing a composition comprising a surfactant, host cells, an aqueous medium and a bio-organic compound produced by the host cells, wherein the solubility of the surfactant in the aqueous medium decreases with increasing temperature and wherein the temperature of the composition is at least about 1° C. below a phase inversion temperature or a cloud point of the composition;

(b) raising the temperature of the composition to at least about 1° C. above the phase inversion temperature or the cloud point; and

(c) performing a liquid/liquid separation of the composition to provide a crude bio-organic composition.

Any surfactant having a solubility in an aqueous medium (e.g., water or a liquid comprising water) that decreases with increasing temperature can be used herein. In certain embodiments, the surfactant is or comprises a non-ionic surfactant. In some embodiments, the non-ionic surfactant is or comprises a polyether polyol, a polyoxyethylene C₈₋₂₀-alkyl ether, a polyoxyethylene C₈₋₂₀-alkylaryl ether (e.g., polyoxyethylene C₈₋₂₀-alkylphenyl ether), a polyoxyethylene C₈₋₂₀-alkyl amine, a polyoxyethylene C₈₋₂₀-alkenyl ether, a polyoxyethylene C₈₋₂₀-alkenyl amine, a polyethylene glycol alkyl ether or a combination thereof. Some non-limiting examples of suitable polyoxyethylene C₈₋₂₀-alkyl ethers include polyoxyethylene lauryl ether, polyoxyethylene cetyl ether, polyoxyethylene stearyl ether, polyoxyethylene branched decyl ether, polyoxyethylene tridecyl ether or a combination thereof. Some non-limiting examples of suitable polyoxyethylene C₈₋₂₀-alkylaryl ethers include polyoxyethylene dodecylphenyl ether, polyoxyethylene nonylphenyl ether, polyoxyethylene octylphenyl ether or a combination thereof. One non-limiting example of suitable polyoxyethylene C₈₋₂₀-alkenyl ether is polyoxyethylene oleic ether. Some non-limiting examples of suitable polyoxyethylene C₈₋₂₀-alkyl amines include polyoxyethylene lauryl amine, polyoxyethylene stearyl amine, polyoxyethylene tallow amine or a combination thereof. One non-limiting example of suitable polyoxyethylene C₈₋₂₀-alkenyl amine is polyoxyethylene oleyl amine. In other embodiments, the non-ionic surfactant is a polyether polyol, polyoxyethylene nonylphenyl ether, polyoxyethylene dodecylphenyl ether or a combination thereof. In certain embodiments, the non-ionic surfactant contains a polyoxyethylene hydrophilic tail.

A phase inversion of a composition or an emulsion occurs when the continuous and dispersed phases of the emulsion are inverted (e.g., an oil-in-water emulsion becomes a water-in-oil emulsion, and vice versa). The temperature at which such a phase inversion occurs is the phase inversion temperature (PIT) of the composition or emulsion. In some embodiments, this phenomenon occurs for a composition or an emulsion containing a surfactant, an aqueous medium and an oil (such as a bio-organic compound disclosed herein), wherein the surfactant has a solubility in the aqueous medium decreasing with increasing temperature. The phase inversion may occur when the temperature is raised to a point where the interaction between water and the surfactant molecules decreases and the surfactant partitioning in water decreases. As a result, the surfactant molecules begin to partition in the oil phase beyond the phase inversion temperature (PIT).

The PIT of a composition or an emulsion may depend on a number of physical, chemical and geometric factors. In general, the PIT can be affected by the physical properties of the liquid components in the composition or emulsion. Some non-limiting examples of such physical properties include viscosity, density and interfacial tension. In some embodiments, the PIT of the composition or emulsion disclosed herein is adjusted, decreased or increased by varying one or more of the physical properties disclosed herein.

The PIT of a composition or an emulsion generally can also be affected by the geometric factors of the vessel that contains and/or processes the composition or emulsion. Some non-limiting examples of such geometric factors include the agitation speed, the number and type of impellers or mixers, the materials of construction and their wetting characteristics. In some embodiments, the PIT of the composition or emulsion disclosed herein is adjusted, decreased or increased by varying one or more of the geometric factors disclosed herein.

The PIT of a composition or an emulsion generally can also be affected by the chemical properties of the components in the composition or emulsion. Some non-limiting examples of the factors are (1) the nature of the hydrophilic and lipophilic moieties of the surfactant; (2) the mixing of the surfactants; (3) the nature of the oil; (4) the nature of the additives of the oil and water phases; (5) the concentration of the surfactant; (6) the ratio of oil phase to water phase, and (7) the distribution of the chain length of the hydrophilic moieties (e.g., the oxyethylene moiety in polyoxyethylene alkyl ethers) in the surfactant. Some of these factors are described in Mitsui et al., Bulletin of the Chemical Society of Japan, Vol. 43, No. 10, 3044-3048 (1970), which is incorporated herein by reference. In some embodiments, the PIT of the composition or emulsion disclosed herein is adjusted, decreased or increased by varying one or more of the chemical properties disclosed herein.

The nature of the hydrophilic and lipophilic moieties of the surfactant may affect the PIT. In general, the PIT increases with an increase in the hydrophilic-lipophilic balance (HLB) value of the surfactant in the composition or emulsion. The HLB value of a surfactant is generally determined by calculating values for the hydrophilic and/or lipophilic regions of the molecule. It is a measure of the degree to which the surfactant is hydrophilic or lipophilic. The HLB values of the surfactants disclosed herein can be measured by any method known in the literature, such as the articles by W. C. Griffin, “Calculation of HLB Values of Non-Ionic Surfactants,” Journal of the Society of Cosmetic Chemists 5:259 (1954); and J. T. Davies, “A quantitative kinetic theory of emulsion type, I. Physical chemistry of the emulsifying agent,” Proceedings of the International Congress of Surface Activity, pp. 426-438 (1957), both of which are incorporated herein by reference.

In some embodiments, the surfactant disclosed herein has a HLB value from about 2 to about 16, from about 2.5 to about 15, from about 3 to about 14, from about 3 to about 10, from about 3 to about 8, or from about 3 to about 6. In certain embodiments, the surfactant has a HLB value from about 4 to about 18, from about 4 to about 16, from about 4 to about 14, from about 4 to about 12, from about 4 to about 10, or from about 4 to about 8. In other embodiments, the surfactant has a HLB value from about 6 to about 18, from about 8 to about 18, from about 8 to about 16, from about 8 to about 14 or from about 8 to about 12. In certain embodiments, the surfactant has a HLB value from about 10 to about 18, from about 12 to about 18 or from about 13 to about 15.

The nature of the oil may affect the PIT of the composition or emulsion comprising the oil. In general, the PIT increases with increasing lipophilicity of the oil. Lipophilicity is generally expressed either by log P or log D. Log P refers to the logarithm of the partition coefficient, P, which is defined as the ratio of the concentration of neutral species in octanol to the concentration of the neutral species in water. Log D refers to the logarithm of the distribution coefficient, D, which is defined as the ratio of the concentration of all species, both neutral and charged, in octanol to the concentration of the all species in water. The lipophilicity of an oil such as the bio-organic compounds disclosed herein can be measured by any method known in the literature. For example, the partition coefficient of the oil can be measured according to ASTM E1147-92, which is incorporated herein by reference. Alternatively, the lipophilicity is determined by the conventional shake-flask method as described in Abraham et al., “Hydrogen bonding. Part 9. The partition of solutes between water and various alcohols,” Phys. Org. Chem., 7:712-716 (1994), which is incorporated herein by reference. In some embodiments, the log P or log D value of the bio-organic compounds disclosed herein is from about 1 to about 6, from about 1 to about 5, from about 1 to about 4 or from about 1 to about 3.

The presence and the nature of the additives of the oil and water phases may affect the PIT of the composition or emulsion. Optionally, the composition or emulsion disclosed herein can comprise one or more additives. Any additive that can be used to adjust, decrease or increase the PIT can be used herein. Some non-limiting examples of additives include water soluble salts and oil soluble components such as paraffins, waxes, organic alcohols and organic acids. In general, nonpolar paraffins and waxes increase the PIT whereas polar organic alcohols and organic acids decrease the PIT.

The concentration of the surfactant may affect the PIT of the composition or emulsion. In general, the PIT decreases with an increase in the concentration of the surfactant. In some embodiments, the concentration of the surfactant is at least about 0.01%, about 0.1%, about 0.25%, about 0.5%, about 0.75%, about 1%, about 2%, about 3%, about 4%, about 5%, about 6%, about 7%, about 8%, about 9%, about 10%, about 15% or about 20% by weight (or by volume), based on the total weight (or volume) of the composition or emulsion. In certain embodiments, the concentration of the surfactant is at most about 0.01%, about 0.1%, about 0.25%, about 0.5%, about 0.75%, about 1%, about 2%, about 3%, about 4%, about 5%, about 6%, about 7%, about 8%, about 9%, about 10%, about 15% or about 20% by weight (or by volume), based on the total weight (or volume) of the composition or emulsion.

The ratio of oil phase to water phase may affect the PIT of the composition or emulsion. In general, the PIT increases with an increase in the ratio of oil phase to water phase. Furthermore, the lower the concentration of the surfactant, the rate of the increase in the PIT is higher. In some embodiments, the ratio of oil phase to water phase is from about 1:100 to about 100:1, from about 1:100 to about 100:1, from about 1:50 to about 50:1, from about 1:20 to about 20:1, from about 1:10 to about 10:1, from about 1:8 to about 8:1, from about 1:6 to about 6:1, from about 1:5 to about 5:1, from about 1:4 to about 4:1, from about 1:3 to about 3:1 or from about 1:2 to about 2:1.

The distribution of the chain length of the hydrophilic moieties in the surfactant may affect the PIT of the composition or emulsion. In general, the PIT decreases with a decrease in the chain length of the hydrophilic moieties (e.g., the oxyethylene moiety in polyoxyethylene alkyl ethers or poly(ethylene oxide) alkylaryl ethers). In some embodiments, the surfactant is a polyoxyethylene alkyl ether or a polyoxyethylene alkylaryl ether. In certain embodiments, the number of oxyethylene units in the polyoxyethylene alkyl ether or polyoxyethylene alkylaryl ether is from about 2 to about 20, from about 3 to about 18, from about 4 to about 16, from about 4 to about 14, from about 4 to about 12, from about 4 to about 10 or from about 4 to about 8.

The PIT of the composition or emulsion disclosed herein can be measured by any method known to a skilled artisan. In some embodiments, the PIT can be determined by observation with the naked eye the temperature at which a phase inversion occurs. In certain embodiments, the PIT can be determined by measuring the pH of the composition or emulsion. In some embodiments, the PIT can be determined by measuring the conductivity of the composition or emulsion. In general, there is an observable change or transition point in appearance, pH or conductivity or other properties of the composition or emulsion at the PIT. Some non-limiting examples of methods for determining the PIT of the composition or emulsion are described in Shinoda et al., “The Correlation between Phase Inversion Temperature in Emulsion and Cloud Point in Solution of Nonionic Emulsifier,” The Journal of Physical Chemistry, Vol. 68, No. 12, 3485-3490 (1964); and Mitsui et al., “An Application of the Phase-inversion-temperature Method to the Emulsification of Cosmetics. I. Factors Affecting the Phase-inversion Temperature,” Bulletin of the Chemical Society of Japan, Vol. 43, No. 10, 3044-3048 (1970), both of which are incorporated herein by reference.

The phase inversion temperature or the cloud point of the composition or emulsion can be controlled or adjusted by one or more physical, chemical and geometric factors disclosed herein. Any phase inversion temperature that is suitable for the methods disclosed herein can be used. In some embodiments, the phase inversion temperature or the cloud point of the composition or emulsion is from about 20° C. to about 90° C., from about 25° C. to about 85° C., from about 30° C. to about 80° C., from about 35° C. to about 75° C., from about 40° C. to about 70° C. or from about 40° C. to about 60° C.

In some embodiments, particularly when the PIT is either unknown or difficult to determine, the cloud point of the surfactant being used can be used instead of the PIT as it can act as a good approximation of the PIT of the composition, as described in Shinoda et al. mentioned above. The cloud point of a surfactant can be measured by any method known to a skilled artisan. In some embodiments, the cloud point of a surfactant is measured by observing with naked eyes the temperature at which a cloudy appearance occurs. In certain embodiments, the cloud point of a surfactant is measured by ASTM D2024-09, titled “Standard Test Method for Cloud Point of Nonionic Surfactants,” which is incorporated herein by reference. In some embodiments, the cloud point is measured by ASTM D2024-09 at a concentration from about 0.1 wt. % to about 1.0 wt. % in deionized water from about 20° C. to about 95° C. In further embodiments, the cloud point is measured by ASTM D2024-09 at a concentration of about 0.5 wt. % or about 1.0 wt. % in deionized water.

The composition or emulsion can be an oil-in-water emulsion or a water-in-oil emulsion, depending on the temperature of the composition or emulsion. In some embodiments, the temperature of the composition or the chemically defined emulsion is below the phase inversion temperature or the cloud point of the composition or emulsion. In certain embodiments, the composition or emulsion is an oil-in-water emulsion wherein its temperature is below its phase inversion temperature or cloud point. In certain embodiments, the temperature of the composition or emulsion is at least about 1° C. below the phase inversion temperature or cloud point of the composition or emulsion. In other embodiments, the temperature of the composition or emulsion is at least about 5° C., at least about 10° C., at least about 15° C., at least about 20° C., at least about 25° C., at least about 30° C., at least about 35° C. or at least about 40° C. below the phase inversion temperature or the cloud point of the composition or emulsion.

In some embodiments, the temperature of the composition or chemically-defined emulsion is above the phase inversion temperature or the cloud point of the composition or emulsion. In certain embodiments, the composition or emulsion is a water-in-oil emulsion wherein its temperature is above its phase inversion temperature or the cloud point. In some embodiments, the temperature of the composition or emulsion is at least about 5° C., at least about 10° C., at least about 15° C., at least about 20° C., at least about 25° C., at least about 30° C., at least about 35° C. or at least about 40° C. above the phase inversion temperature or the cloud point of the composition or emulsion.

In another aspect, provided herein are methods that comprise:

(a) providing an oil-in-water emulsion comprising a surfactant, host cells, an aqueous medium and a bio-organic compound produced by the host cells, wherein the solubility of the surfactant in the aqueous medium decreases with increasing temperature;

(b) converting the oil-in-water emulsion to a water-in-oil emulsion; and

(c) performing a liquid/liquid separation of the water-in-oil emulsion to provide a crude bio-organic composition.

The conversion of an oil-in-water emulsion to the corresponding water-in-oil emulsion can be effected by any method known in the literature. In some embodiments, the conversion is effect by raising the temperature of the oil-in-water emulsion to a temperature above its PIT. In certain embodiments, the conversion is effect by (1) keeping the temperature of the oil-in-water emulsion at a particular temperature or in a range of temperature; and (2) reducing the PIT of the oil-in-water emulsion to a value below the particular temperature or the range of temperature using one or more physical, chemical and geometric factors disclosed herein. In other embodiments, the conversion is effect by (1) raising or lowering the temperature of the oil-in-water emulsion to a particular temperature or a range of temperature; and (2) adjusting the PIT of the oil-in-water emulsion to a value below the particular temperature or the range of temperature using one or more physical, chemical and geometric factors disclosed herein.

In certain embodiments, the bio-organic compound is a hydrocarbon. In certain embodiments, the bio-organic compound is a C₅-C₃₀ hydrocarbon. In certain embodiments, the bio-organic compound is an isoprenoid. In further embodiments, the bio-organic compound is a C₅-C₂₀ isoprenoid. In additional embodiments, the bio-organic compound is a C₁₀-C₁₅ isoprenoid. In certain embodiments, the bio-organic compound is a fatty acid or a fatty acid derivative. In certain embodiments, the bio-organic compound is a C₅-C₃₅ fatty acid or a fatty acid derivative. In additional embodiments, the bio-organic compound is selected from carene, geraniol, linalool, limonene, myrcene, ocimene, pinene, sabinene, terpinene, terpinolene, amorphadiene, farnesene, farnesol, nerolidol, valencene and geranylgeraniol or a combination thereof. In further additional embodiments, the bio-organic compound is myrcene, α-ocimene, β-ocimene, α-pinene, β-pinene, amorphadiene, α-farnesene, β-farnesene or a combination thereof. In certain embodiments, the bio-organic compound is α-farnesene, β-farnesene or a mixture thereof.

In certain embodiments, the microbial cells are bacteria. In certain embodiments, the microbial cells belong to the genera Escherichia, Bacillus, Lactobacillus. In certain embodiments, the microbial cells are E. coli. In further embodiments, the microbial cells are fungi. In still further embodiments, the microbial cells are yeast. In still further embodiments, the microbial cells are Kluyveromyces, Pichia, Saccharomyces and Yarrowia. In additional embodiments, the microbial cells are S. cerevisiae. In certain embodiments, the microbial cells are algae. In certain embodiments, the microbial cells are Chlorella minutissima, Chlorella emersonii, Chloerella sorkiniana, Chlorella ellipsoidea, Chlorella sp. or Chlorella protothecoides.

In certain embodiments, the clarifying step occurs by liquid/solid separation. In other embodiments, the clarifying step occurs by sedimentation followed by decantation. In still other embodiments, the clarifying step occurs by filtration. In certain embodiments, the clarifying step occurs by centrifugation. In certain other embodiments, the clarifying step occurs in a continuous disk stack nozzle centrifuge.

Optionally, the pH of the composition or emulsion can be adjusted to a pH greater than about 7.5. In certain embodiments, the pH of the composition or emulsion is adjusted to a pH between about 7.5 and about 10. In some embodiments, the pH of the composition or emulsion is adjusted to a pH between about 7.5 and about 9. In other embodiments, the pH of the composition or emulsion is adjusted to a pH between about 8 and about 8.5. In some embodiments, the pH of the composition or emulsion is adjusted to a pH greater than 9.

The pH of the composition or emulsion can be adjusted by using any base deemed suitable by one of skill in the art. Illustrative examples of suitable bases include: ammonia, potassium hydroxide, barium hydroxide, cesium hydroxide, sodium hydroxide, strontium hydroxide, calcium hydroxide, lithium hydroxide, rubidium hydroxide and magnesium hydroxide. Highly soluble and economical bases are generally preferred for commercial scale operations. Illustrative examples of such bases include potassium hydroxide and sodium hydroxide.

In certain embodiments, the composition or emulsion is separated by liquid/liquid separation. In certain embodiments, the composition or emulsion is separated by centrifugation that relies on the different densities between the bio-organic compound and the aqueous medium. In certain embodiments, the composition or emulsion is separated by a continuous disk-stack centrifugation. In certain embodiments, the composition or emulsion is separated by liquid/liquid extraction (also known as solvent extraction).

In certain embodiments, the method further comprises concentrating the bio-organic compound in the composition or emulsion into a concentrated composition or emulsion thereby reducing the volume for subsequent downstream processing. Thus, if the concentration step occurs, then the pH adjustment step and the liquid-liquid separation step are performed on the concentrated composition or emulsion instead of on the composition or emulsion.

Thus in another aspect, the methods comprise:

(a) providing a first composition comprising a surfactant, host cells, an aqueous medium and a bio-organic compound produced by the host cells, wherein the solubility of the surfactant in the aqueous medium decreases with increasing temperature;

(b) concentrating the first composition to form a concentrated composition wherein the concentrated composition comprises substantially all of the bio-organic compound and the volume of the concentrated composition is less than the volume of the first composition, wherein the temperature of the concentrated composition is at least about 1° C. below a phase inversion temperature or a cloud point of the concentrated composition;

(c) raising the temperature of the concentrated composition to at least about 1° C. above the phase inversion temperature or the cloud point; and

(d) performing a liquid/liquid separation of the concentrated composition to provide a crude bio-organic composition.

In another aspect, provided herein are methods that comprise:

(a) providing a first oil-in-water emulsion comprising a surfactant, host cells, an aqueous medium and a bio-organic compound produced by the host cells, wherein the solubility of the surfactant in the aqueous medium decreases with increasing temperature;

(b) concentrating the first oil-in-water emulsion to form a concentrated oil-in-water emulsion wherein the concentrated oil-in-water emulsion comprises substantially all of the bio-organic compound and the volume of the concentrated oil-in-water emulsion is less than the volume of the first oil-in-water emulsion;

(c) converting the concentrated oil-in-water emulsion to a water-in-oil emulsion; and

(d) performing a liquid/liquid separation of the water-in-oil emulsion to provide a crude bio-organic composition.

In certain embodiments, the concentrated composition or emulsion comprises about 50 percent of the volume of the first composition or emulsion. In certain embodiments, the concentrated composition or emulsion is at most about 40, 35, 30, 25, 20, 15, 10, 5, 4, 3, 2 or 1 percent of the volume of the first composition or emulsion. In certain embodiments, the concentrated composition or emulsion is at most about 25 percent of the volume of the first composition or emulsion. In further embodiments, the concentrated composition or emulsion is at most about 10 percent of the volume of the first composition or emulsion. In still further embodiments, the concentrated composition or emulsion is at most about 5 percent of the volume of the first composition or emulsion.

In certain embodiments, the concentration step occurs by tangential flow filtration (“TFF”). For example the clarified composition or emulsion (which is substantially free of host cells) is dewatered using TFF to produce a concentrated composition or emulsion. In certain other embodiments, the clarification and concentration steps occur simultaneously. For example, when the clarifying step occurs by sedimentation of the host cells, the top portion of the mixture, containing substantially all of the bio-organic compound, can be decanted. This top layer then becomes the concentrated composition or emulsion. In another example, if the clarifying step occurs from using a continuous disk stack nozzle centrifuge, then the portion of the mixture that includes the bio-organic compound can be separated based on the different densities between the bio-organic compound and the aqueous medium. The portion containing the bio-organic compound then becomes the concentrated composition or emulsion.

Optionally, the pH of the concentrated composition or emulsion can be adjusted to a pH greater than about 7.5. In certain embodiments, the pH of the concentrated composition or emulsion is adjusted to a pH between about 7.5 and about 10. In certain embodiments, the pH of the concentrated composition or emulsion is adjusted to a pH between about 7.5 and about 9. In certain embodiments, the pH of the concentrated composition or emulsion is adjusted to a pH between about 8 and about 8.5. In additional embodiments, the pH of the concentrated composition or emulsion is adjusted to a pH greater than 9.

In certain embodiments, the concentrated composition or emulsion is separated by liquid/liquid separation to provide a crude bio-organic composition. In certain embodiments, the concentrated composition or emulsion is separated by centrifugation that relies on the different densities between the bio-organic compound and the aqueous medium. In certain embodiments, the concentrated composition or emulsion is separated by a continuous, three-phase, disk-stack centrifugation. In certain embodiments, the concentrated composition or emulsion is separated by liquid/liquid extraction (also known as solvent extraction).

In certain embodiments, the method further comprises purifying the crude bio-organic composition to yield a purified bio-organic composition. Any suitable method may be used and is likely to depend on the desired level of purity of the bio-organic compound or the acceptable levels of impurities in the final composition. Suitable methods include, but are not limited to: fractional distillation, adsorption and liquid chromatography. In certain embodiments, the purification is by flash distillation. In certain embodiments, the purification is by silica gel filtration. In additional embodiments, the purification is by alumina filtration.

In another aspect, the methods comprise:

(a) providing a first composition or an emulsion comprising a surfactant, host cells, an aqueous medium and a bio-organic compound produced by the host cells, wherein the solubility of the surfactant in the aqueous medium decreases with increasing temperature;

(b) concentrating the first composition or emulsion to form a concentrated composition or emulsion wherein the concentrated composition or emulsion comprises substantially all of the bio-organic compound and the volume of the concentrated composition or emulsion is less than the volume of the first composition or emulsion, wherein the temperature of the concentrated composition or emulsion is at least about 1° C. below a phase inversion temperature or a cloud point of the concentrated composition or emulsion;

(c) raising the temperature of the concentrated composition or emulsion to at least about 1° C. above the phase inversion temperature or the cloud point;

d) centrifuging the concentrated composition or emulsion to separate the bio-organic compound from the aqueous medium thereby forming a crude bio-organic composition; and

e) flash distilling the neutralized crude composition to yield a neutralized purified composition.

In certain embodiments, the host cells are yeast cells.

In certain embodiments, the purified composition (whether neutralized or not) is further polished. For example, when the bio-organic compound is an olefin, the method can further comprise adding an antioxidant to the purified bio-organic composition. The addition of the antioxidant can retard the formation of peroxides and stabilizes the purified bio-organic composition. Any anti-oxidant deemed suitable by one of skill in the art can be used. However, if the olefin is to be subsequently hydrogenated, a phenolic antioxidant which does not interfere with hydrogenation reactions under mild conditions like certain commonly used antioxidants such as α-tocopherol is preferred. Illustrative examples of suitable anti-oxidants include: resveratrol; 3-tert-butyl-4-hydroxyanisole; 2-tert-butyl-4-hydroxyanisole; 2,4-dimethyl-6-tert-butylphenol; 2,6-di-tert-butyl-4-methylphenol; and 4-tert-butylcatechol.

In another example, the purified compositions can be further polished by the addition of a chelating agent to reduce the amounts of metals in the compositions. In certain embodiments, the purification step also includes removing metals present in the crude bio-organic composition by the addition of a chelating agent. Any suitable chelating agent can be used. Illustrative examples of suitable chelating agents include ascorbic acid, citric acid, malic acid, oxalic acid, succinic acid, dicarboxymethyllutamic acid, ethylenediaminedisuccinic acid (EDDS), ethylenediaminetetraacetic acid (EDTA) and the like.

While the processes and systems provided herein have been described with respect to a limited number of embodiments, the specific features of one embodiment should not be attributed to other embodiments of the processes or systems. No single embodiment is representative of all aspects of the methods or systems. In certain embodiments, the processes may include numerous steps not mentioned herein. In other embodiments, the processes do not include any steps not enumerated herein. Variations and modifications from the described embodiments exist.

It is noted that the purification methods are described with reference to a number of steps. In certain embodiments, these steps can be practiced in any sequence. In certain embodiments, one or more steps may be omitted or combined but still achieve substantially the same results. The appended claims intend to cover all such variations and modifications as falling within the scope of the claimed subject matter.

All publications and patent applications mentioned in this specification are herein incorporated by reference to the same extent as if each individual publication or patent application was specifically and individually indicated to be incorporated by reference. Although the claimed subject matter has been described in some detail by way of illustration and example for purposes of clarity of understanding, it will be readily apparent to those of ordinary skill in the art in light of the teachings herein that certain changes and modifications may be made thereto without departing from the spirit or scope of the appended claims.

### EXAMPLES

### Example 1

### Preparation of CCB

This example describes a method for preparing concentrated, clarified broth (hereafter “CCB”).

A fermentation harvest broth from pilot plant fermentations was fractionated using continuous centrifugation in a pilot scale, continuous nozzle centrifuge. Two output streams (concentrate and centrate) were produced. The concentrate stream containing sedimented cells and aqueous waste was discharged from the nozzles. From the centrate stream, CCB containing about 50% water and about 50% farnesene was collected. Each fermentation lot was given a unique lot number based on the inoculation date.

### Example 2

### Effect of Different Surfactant Concentrations on Farnesene Released from Cane Syrup Derived CCB at 60° C.

This example shows the effect of different surfactants, including TERGITOL™ L62 and TERGITOL™ L64, on farnesene release or the amount of farnesene released (in term of oil recovery and oil release rate) from cane syrup derived CCB at incubation temperature of 60° C.

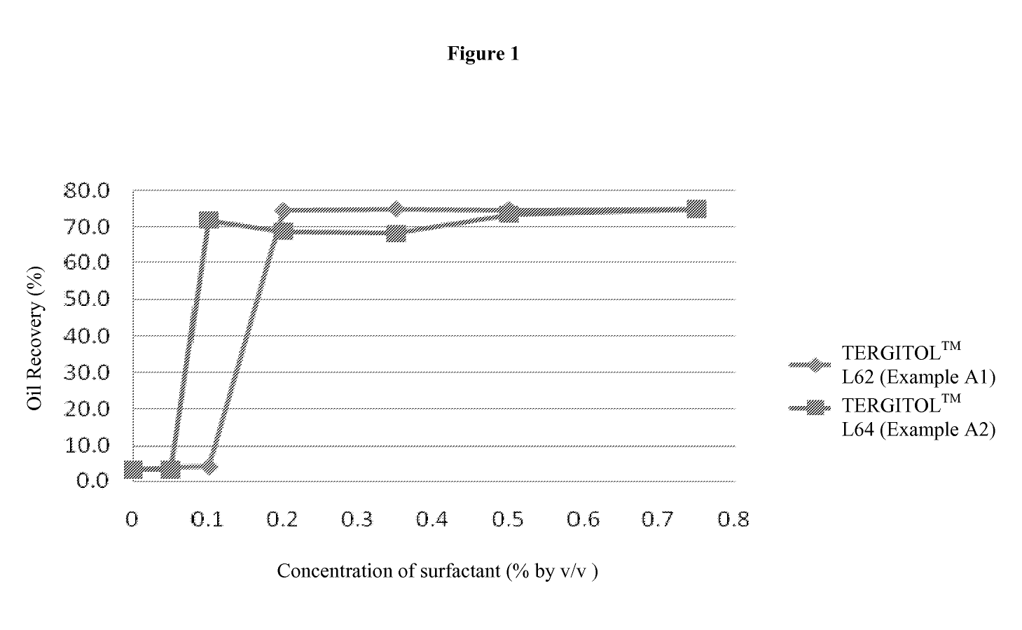

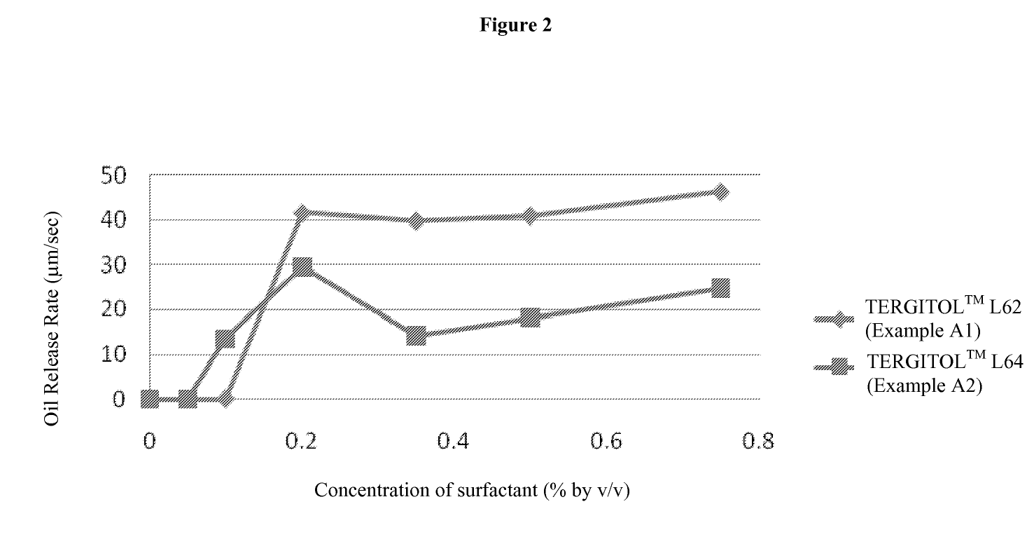

CCB (Lot No.: PP031910F2_draw2) (1 ml per tube) was aliquoted into 1.5 ml microcentrifuge tubes. Different concentrations of TERGITOL™ L62 or TERGITOL™ L64 were added into the tubes. The contents of each tube were then mixed at ambient temperature for 10 minutes by a vortex mixer. The tubes were then incubated in a hot bath at about 60° C. for 30 minutes. Samples (400 μl) from the tubes were added into lumisizer microcentrifuge cells and analyzed by HIGH-END DISPERSION ANALYSER LUMISIZER®, an analytical centrifuge commercially obtained from L. U. M. GmbH, Berlin, Germany, (hereafter “the Lumisizer”). The samples in the Lumisizer were centrifuged at 4000 rpm (2300×g) at about 60° C. for 22 minutes. In order to prevent heat loss during the transfer of the samples into the cells, each cell was placed into a hot bath at about 60° C. until the transferring step was completed. The samples with TERGITOL™ L62 were labeled as Example A1, whereas samples with TERGITOL™ L64 were labeled as Example A2. The oil recovery and oil release rate of Examples A1-A2 were determined and plots of the oil recovery and oil release rate versus the concentration of the surfactants are shown in FIG. 1 and FIG. 2 respectively.

Referring to FIG. 1, there were sharp increases in the oil recovery with an increase in the concentrations of the TERGITOL™ L62 and TERGITOL™ L64 respectively. This indicated that there was a critical threshold concentration for emulsion breakage. Referring to FIG. 2, Example A1 has a higher oil release rate than that of Example A2. This indicated that TERGITOL™ L62 released more oil (i.e., farnesene) from cane syrup derived CCB at 60° C. than TERGITOL™ L64.

### Example 3

### Comparison of Oil Recovery and Oil Release Rate Using Different Surfactants, Including TERGITOL™ L62, TERGITOL™ L64, ECOSURF™ SA-7 and ECOSURF™ SA-9

This example shows the effect of different surfactants, including TERGITOL™ L62, TERGITOL™ L64, ECOSURF™ SA-7 and ECOSURF™ SA-9, on the amount of farnesene released (in term of oil recovery and oil release rate) from cane syrup derived CCB at incubation temperature of 60° C.

Surfactants having similar cloud points but different chemical structures were tested for demulsifying CCB. The surfactants used here include TERGITOL™ L62, TERGITOL™ L64, ECOSURF™ SA-7 and ECOSURF™ SA-9.

CCB (Lot No.: PP040210F2_draw1) (1 ml per tube) was aliquoted into 1.5 ml microcentrifuge tubes. Different concentrations of different surfactants were added into the tubes. The contents of each tube were then mixed at ambient temperature for 10 minutes by a vortex mixer. The tubes were then incubated in a hot bath at about 70° C. for approximately an hour. Samples (400 μl) from the tubes were added into lumisizer microcentrifuge cells and analyzed by the Lumisizer. The samples in the Lumisizer were centrifuged at 4000 rpm (2300×g) at about 60° C. for 22 minutes. In order to prevent heat loss during the transfer of the samples into the cells, each cell was placed into a hot bath at about 60° C. until the transferring step was completed.

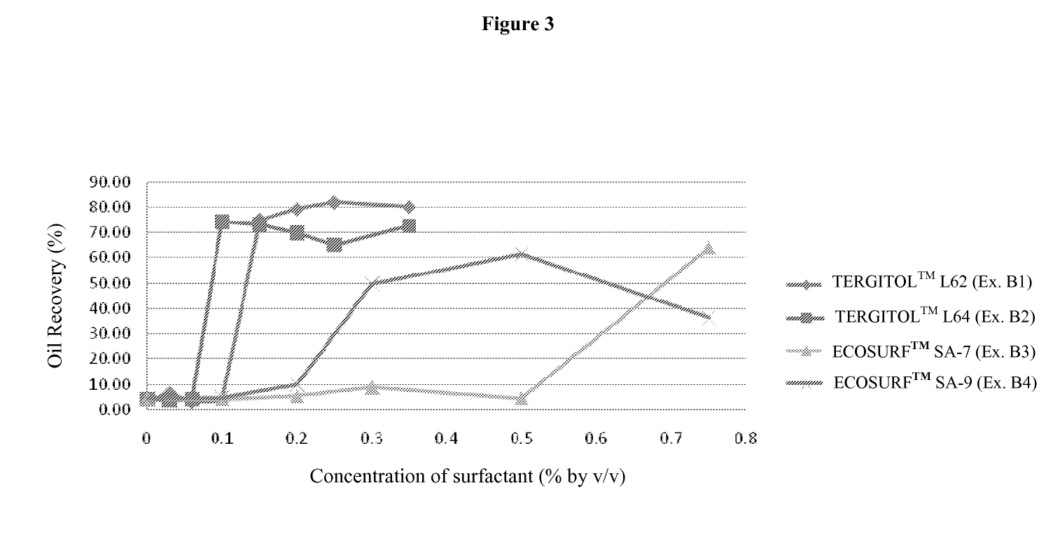

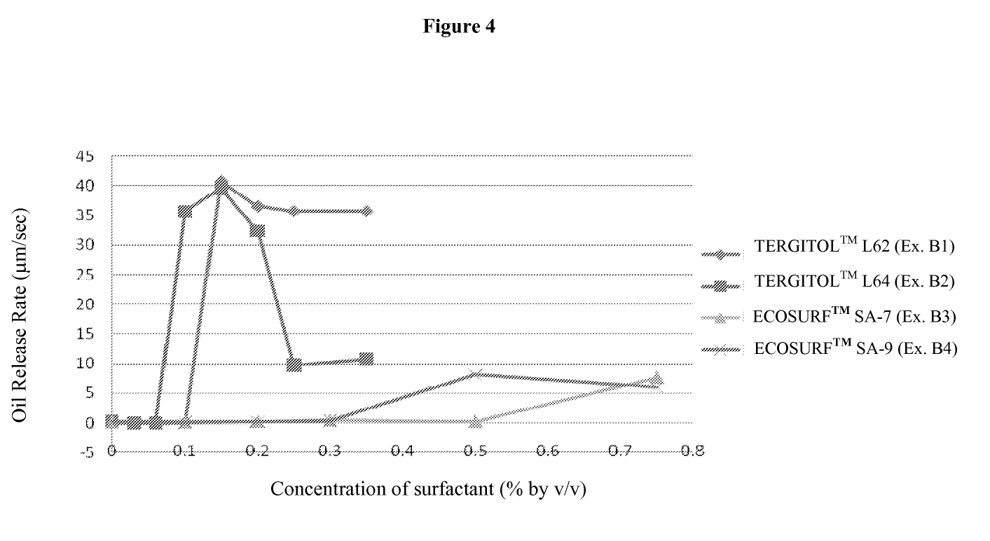

The oil recovery and oil release rate of each sample were determined and plots of the oil recovery and oil release rate versus the concentration of the surfactants were shown in FIGS. 3 and 4 respectively, where the samples with TERGITOL™ L62 were labeled as Example B1; the samples with TERGITOL™ L64 were labeled as Example B2; the samples with ECOSURF™ SA-7 were labeled as Example B3; and the samples with ECOSURF™ SA-9 were labeled as Example B4.

Titration curves obtained from Examples B1 and B2 were different from the curves obtained from Examples B3 and B4. The curves of Example B1 and Example B2 had a sharp increase in the oil recovery, whereas each of Examples B3 and B4 had a more gradual response in the oil recovery when the concentration of surfactant increased.

The oil recoveries of Examples B1 and B2 were higher than those of Examples B3 and B4 at low concentrations of surfactant. The data shows that 0.2% by v/v or less of TERGITOL™ L62 or TERGITOL™ L64 was sufficient to release farnesene from CCB.

TERGITOL™ L62 and TERGITOL™ L64 (obtained from The Dow Chemical Company, Midland, Mich.) are polyether polyol, nonionic surfactants which are chemically synthesized compounds, whereas ECOSURF™ SA-7 and ECOSURF™ SA-9 (obtained from The Dow Chemical Company, Midland, Mich.) are modified alcohol ethoxylate based, nonionic surfactants which are modified from natural sourced seed oils.

### Example 4

### Effect of Surfactant Concentration on Farnesene Released from Cane Syrup Derived CCB at 30° C. and 40° C.

This example shows the effect of the concentration of different surfactants, including TERGITOL™ L64, TERGITOL™ NP-7, and TERGITOL™ TMN-6, on farnesene release or the amount of farnesene released from cane syrup derived CCB at incubation temperatures of 30° C. and 40° C.

CCB (Lot No.: PP040910F1) (1 ml per tube) was aliquoted into 1.5 ml microcentrifuge tubes. Different concentrations of surfactants were added into the tubes. The contents of each tube were then mixed at ambient temperature for about 10 minutes by a vortex mixer. The tubes were then incubated at 30° C. and 40° C. respectively for about 15 minutes. After incubation, the tubes were centrifuged at 10,000×g at the incubation temperatures for 5 minutes.

The tubes incubated at 40° C. with TERGITOL™ L64 in an amount ranged from 0.1% to 0.4% by v/v were labeled as Examples C1-C4 respectively. The tubes incubated at 40° C. with 0.2 vol. % and 0.5 vol. % of TERGITOL™ NP-7 were labeled as Examples C5-C6 respectively. The tubes incubated at 40° C. with 0.2 vol. % and 0.5 vol. % of TERGITOL™ TMN-6 were labeled as Examples C7-C8 respectively. The tubes incubated at 30° C. with TERGITOL™ L64 ranged from 0.1% to 0.4% by v/v were labeled as Examples C9-C12 respectively. The tubes incubated at 30° C. with 0.2 vol. % and 0.5 vol. % of TERGITOL™ NP-7 were labeled as Examples C13-C14 respectively. The tubes incubated at 30° C. with 0.2 vol. % and 0.5 vol. % of TERGITOL™ TMN-6 were labeled as Examples C15-C16 respectively.

Two control experiments (i.e., Controls C1-C2) were done at 30° C. and 40° C. respectively according to the same procedure above except without the addition of a surfactant. Table 2 below provides the conditions for Examples C1-C16 and Controls C1-C2.

By observing the samples after centrifugation, it was found that there were 2 layers (an aqueous phase bottom layer and an emulsified farnesene top layer) in Controls C1-C2 whereas there were 3 layers (an aqueous phase bottom layer, an emulsified farnesene middle layer and a clear farnesene top layer) in Examples C1-C16. The amount of the clear farnesene top layer in Examples C6 and C14 were observed to be highest among all samples. Therefore, the TERGITOL™ NP-7 in Examples C6 and C14 was found to be highly effective in releasing farnesene from the cane syrup derived CCB at a temperature as low as 30° C., which was consistent with the cloud point (20° C.) of the TERGITOL™ NP-7 used. The amount of the clear farnesene top layer in Example C8 was about the same as those in Examples C6 and C14. However, the amount of the clear farnesene top layer in Example C16 was much less than those in Examples C8 and C6 and C14. Therefore, the TERGITOL™ TMN-6 was found to be highly effective in releasing farnesene from the cane syrup derived CCB at a temperature as low as 40° C., although not at 30° C., which was consistent with the cloud point (36° C.) of TERGITOL™ TMN-6. However, the amounts of the clear farnesene top layer in Examples C1-C4 and C9-C12 were much less than those in Examples C8 and C6 and C14. Therefore, TERGITOL™ L-64 was found not effective in releasing farnesene from the cane syrup derived CCB at both 30° C. and 40° C., which was consistent with the cloud point (62° C.) of TERGITOL™ L-64.

TABLE 2
A list of conditions of Examples C1-C16 and Controls C1-C2

| | Incubation | | Concentration |
|---|---|---|---|
| | Temperature | | of Surfactant |
| Sample | (° C.) | Type of Surfactant | (% by v/v) |
| Example C1 | 40 | TERGITOL ™ L64 | 0.1 |
| Example C2 | 40 | TERGITOL ™ L64 | 0.2 |
| Example C3 | 40 | TERGITOL ™ L64 | 0.3 |
| Example C4 | 40 | TERGITOL ™ L64 | 0.4 |
| Example C5 | 40 | TERGITOL ™ NP-7 | 0.2 |
| Example C6 | 40 | TERGITOL ™ NP-7 | 0.5 |
| Example C7 | 40 | TERGITOL ™ TMN-6 | 0.2 |
| Example C8 | 40 | TERGITOL ™ TMN-6 | 0.5 |
| Example C9 | 30 | TERGITOL ™ L64 | 0.1 |
| Example C10 | 30 | TERGITOL ™ L64 | 0.2 |
| Example C11 | 30 | TERGITOL ™ L64 | 0.3 |
| Example C12 | 30 | TERGITOL ™ L64 | 0.4 |
| Example C13 | 30 | TERGITOL ™ NP-7 | 0.2 |
| Example C14 | 30 | TERGITOL ™ NP-7 | 0.5 |
| Example C15 | 30 | TERGITOL ™ TMN-6 | 0.2 |
| Example C16 | 30 | TERGITOL ™ TMN-6 | 0.5 |
| Control C1 | 40 | — | 0 |
| Control C2 | 30 | — | 0 |

### Example 5

### Effect of Incubation Temperatures and Different Surfactants on Farnesene Released from Cane Syrup Derived CCB

This example shows the effect of incubation temperatures at 30° C., 40° C., 50° C. and 60° C. and different surfactants, including TERGITOL™ L62, TERGITOL™ L64, and TRITON™ X114, on the amount of farnesene released from cane syrup derived CCB.

CCB (Lot No.: PP041610F2) (1 ml per tube) was aliquoted into 1.5 ml microcentrifuge tubes. Different surfactants, including TERGITOL™ L62, TERGITOL™ L64 and TRITON™ X114, in an amount of 0.5% by v/v were added into the tubes. The contents of each tube were then mixed at ambient temperature for about 10 minutes by a vortex mixer. The tubes were then incubated at 30° C., 40° C., 50° C. and 60° C. for about 15 minutes respectively. Samples (400 μl) from the tubes was added into lumisizer microcentrifuge cells and analyzed by the Lumisizer. The samples in the Lumisizer were centrifuged at 4000 rpm (2300×g) at the incubation temperatures for 22 minutes.

The samples with 0.5% by v/v of TERGITOL™ L62 incubated at 30° C., 40° C., 50° C. and 60° C. were labeled as Examples D1, D4, D7 and D10 respectively. The samples with 0.5% by v/v of TERGITOL™ L64 incubated at 30° C., 40° C., 50° C. and 60° C. were labeled as Examples D2, D5, D8 and D11 respectively. The samples with 0.5% by v/v of Tritron X114 incubated at 30° C., 40° C., 50° C. and 60° C. were labeled as Examples D3, D6, D9 and D12 respectively. Four control experiments (Controls D1-D4) were carried out according to the procedure as mentioned above except without the addition of a surfactant. Table 3 provides the conditions of Examples D1-D12 and Controls D1-D4.

The oil release rate and oil recovery of Examples D1-D12 and Controls D1-D4 were determined. Tables 3 and 4 provide the oil release rate and oil recovery results for Examples D1-D12 and Controls D1-D4.

TABLE 3
Oil release rates of Examples (D1-D12) and Controls D1-D4

| | | Concentration | Incubation | Oil release |
|---|---|---|---|---|
| | Type of | of Surfactant | Temperature | rates |
| Sample | Surfactant | (% by v/v) | (° C.) | (μm/sec) |
| Control D1 | — | — | 30 | −0.0401 |
| Example D1 | TERGITOL ™ | 0.5 | 30 | 0.0063 |
| | L62 | | | |
| Example D2 | TERGITOL ™ | 0.5 | 30 | −0.0853 |
| | L64 | | | |
| Example D3 | TRITON ™ | 0.5 | 30 | 3.1361 |
| | X114 | | | |
| Control D2 | — | — | 40 | −0.1674 |
| Example D4 | TERGITOL ™ | 0.5 | 40 | 0.1117 |
| | L62 | | | |
| Example D5 | TERGITOL ™ | 0.5 | 40 | −0.1327 |
| | L64 | | | |
| Example D6 | TRITON ™ | 0.5 | 40 | 12.2822 |
| | X114 | | | |
| Control D3 | — | — | 50 | −0.2128 |
| Example D7 | TERGITOL ™ | 0.5 | 50 | 0.2063 |
| | L62 | | | |
| Example D8 | TERGITOL ™ | 0.5 | 50 | 0.0904 |
| | L64 | | | |
| Example D9 | TRITON ™ | 0.5 | 50 | 15.5995 |
| | X114 | | | |
| Control D4 | — | — | 60 | −0.0725 |
| Example | TERGITOL ™ | 0.5 | 60 | 13.7826 |
| D10 | L62 | | | |
| Example | TERGITOL ™ | 0.5 | 60 | 9.9733 |
| D11 | L64 | | | |
| Example | TRITON ™ | 0.5 | 60 | 36.1787 |
| D12 | X114 | | | |

TABLE 4
Oil recovery of Examples (D1-D12) and Controls D1-D4

| | | | | | | % Clear Oil |
|---|---|---|---|---|---|---|
| | | | | | | In Emulsion |
| | | Concentration | Incubation | Emulsion | Clear Oil | (which is |
| | Type of | of Surfactant | Temperature | Length | Length | equal to oil |
| Sample | Surfactant | (% by v/v) | (° C.) | (mm) | (mm) | recovery |
| Control | — | — | 30 | 9.27 | 0.42 | 5% |
| D1 | | | | | | |
| Example | TERGITOL ™ | 0.5 | 30 | 10.01 | 0.7 | 7% |
| D1 | L62 | | | | | |
| Example | TERGITOL ™ | 0.5 | 30 | 9.29 | 0.52 | 6% |
| D2 | L64 | | | | | |
| Example | TRITON ™ | 0.5 | 30 | 9.01 | 0.85 | 9% |
| D3 | X114 | | | | | |
| Control | — | — | 40 | 9.38 | 0.4 | 4% |
| D2 | | | | | | |
| Example | TERGITOL ™ | 0.5 | 40 | 9.99 | 0.7 | 7% |
| D4 | L62 | | | | | |
| Example | TERGITOL ™ | 0.5 | 40 | 9.47 | 0.52 | 5% |
| D5 | L64 | | | | | |
| Example | TRITON ™ | 0.5 | 40 | 8.95 | 7.44 | 83% |
| D6 | X114 | | | | | |
| Control | — | — | 50 | 9.33 | 0.6 | 6% |
| D3 | | | | | | |
| Example | TERGITOL ™ | 0.5 | 50 | 9.25 | 2.2 | 24% |
| D7 | L62 | | | | | |
| Example | TERGITOL ™ | 0.5 | 50 | 9.41 | 1.85 | 20% |
| D8 | L64 | | | | | |
| Example | TRITON ™ | 0.5 | 50 | 8.54 | 8 | 94% |
| D9 | X114 | | | | | |
| Control | — | — | 60 | 9.11 | 0.79 | 9% |
| D4 | | | | | | |
| Example | TERGITOL ™ | 0.5 | 60 | 11.28 | 5.93 | 53% |
| D10 | L62 | | | | | |
| Example | TERGITOL ™ | 0.5 | 60 | 9.02 | 6.3 | 70% |
| D11 | L64 | | | | | |
| Example | TRITON ™ | 0.5 | 60 | 9.08 | 7.85 | 86% |
| D12 | X114 | | | | | |

The oil release rate of the sample (Example D3) with TRITON™ X114 (incubated at 30° C.) was the highest among the samples (Examples D1-D3) with same incubation temperature which was consistent with the cloud point of TRITON™ X114 (25° C.). The oil release rate of the samples with TERGITOL™ L62 and TERGITOL™ L64 increased with the incubation temperature which was consistent with the cloud points of TERGITOL™ L62 and TERGITOL™ L64 at 32° C. and 62° C. respectively.

### Example 6

### Effect of Different Surfactants on the Oil Recovery and Oil Release Rate

This example shows the effect of different surfactants, including TERGITOL™ L62 and TRITON™ X114, on the amount of farnesene released from a defined medium fermentation broth derived CCB at incubation temperature of 50° C.

CCB isolated from the defined media fermentation was aliquoted in an amount of 1 ml per tube into 1.5 ml microcentrifuge tubes. Different surfactants, including TRITON™ X114 in an amount of 0.2% or 0.5% by v/v; and TERGITOL™ L62 in an amount of 0.2% by v/v, were added into the tubes. The contents of each tube were then mixed at ambient temperature for about 10 minutes by a vortex mixer. The tubes were then incubated at about 50° C. for about 15 minutes. Samples (400 μl) from the tubes were added into lumisizer microcentrifuge cells and analyzed by the Lumisizer. The samples in the Lumisizer were centrifuged at 4000 rpm (2300×g) at 50° C. for 22 minutes.

The samples with 0.2% or 0.5% by v/v TRITON™ X114 were labeled as Examples E1 and E2 respectively. The sample with 0.2% by v/v TERGITOL™ L62 was labeled as Example E3.

The oil release rate and oil recovery of each sample were determined. Tables 5 and 6 provide the oil release rate and oil recovery results for Examples E1-E3.

TABLE 5
Oil release rates of Examples E1-E3

| | | Concentration | |
|---|---|---|---|
| | | of Surfactant | Oil release rates |
| Sample | Type of Surfactant | (% by v/v) | (μm/sec) |
| Example E1 | TRITON ™ X114 | 0.2% | 21.7 |
| Example E2 | TRITON ™ X114 | 0.5% | 23.1 |
| Example E3 | TERGITOL ™ L62 | 0.2% | 35.9 |

TABLE 6
Oil recovery of Examples E1-E3

| | | | | | | % Clear Oil |
|---|---|---|---|---|---|---|
| | | Concentration | | Emulsion | Clear Oil | In Emulsion |
| | Type of | of Surfactant | Temp. | Length | Length | (which is equal |
| Sample | Surfactant | (% by v/v) | (° C.) | (mm) | (mm) | to oil recovery) |
| Example | TRITON ™ | 0.2% | 50 | 6.48 | 5.96 | 92% |
| E1 | X114 | | | | | |
| Example | TRITON ™ | 0.5% | 50 | 6.65 | 6.13 | 92% |
| E2 | X114 | | | | | |
| Example | TERGITOL ™ | 0.2% | 50 | 6.6 | 5.9 | 89% |
| E3 | L62 | | | | | |

### Example 7

### Process at Pilot Scale

This example demonstrates the possibility of releasing farnesene from CCB at pilot scale.

Whole cell broth (WCB) was obtained directly from the fermentor. CCB was collected from the centrate as mentioned in Example 1.

TRITON™ X114 (0.2% by v/v) was added to WCB, mixed and heated to 53° C. The mixture was centrifuged at 4000 rpm (2300×g) for 22 minutes at 53° C.

CCB (2.5 L) isolated from a defined media fermentation (20 L) was treated with TRITON™ X114 (0.2% by v/v), mixed and then heated to about 53° C. for 15 minutes. The mixture was centrifuged at 4000 rpm (2300×g) for 22 minutes at 53° C.

The concentration of farnesene was measured by gas chromatography with flame ionization (GC-FID).

Table 7 provides the average concentration of farnesene, step volume and weight of farnesene extracted from WCB, CCB, liquid/liquid aqueous phase and crude farnesene which were labeled as Examples F1, F2, F3 and F4 respectively.

Contracted manufacturing organization (CMO) process was followed:

The pH of each run was titrated to 9.5 with 5N NaOH. NaCl (0.56 M) was added. TERGITOL™ L81 was added and the mixture was mixed for an hour at ambient temperature.

Table 8 provides the conditions, i.e., pH 9.5/0.65M NaCl/0.5% L81, and the concentration of farnesene in the liquid/liquid aqueous phase obtained from samples with different extraction processes, i.e., CMO process (Examples F5-F9) and Example F3. The concentration of farnesene in the liquid/liquid aqueous phase of Examples F5-F9 ranged from 25 g/L to 67 g/L. The concentration of farnesene in the liquid/liquid aqueous phase of Example F3 with 0.2% TRITON™ X114 at 53° C. was merely 5 g/L, which was at least 5 folds reduction compared with those of Examples F5-F9. The data suggest that the TRITON™ X114 process may result in a reduction in farnesene loss across the Liquid/Liquid centrifugation unit operation.

TABLE 7
Average concentration of farnesene, step volume and weight
of farnesene extracted from WCB, CCB, Liquid/liquid Aqueous
Phase and Crude farnesene

| | | Average | | |
|---|---|---|---|---|
| | | Concentration | | |
| | | of Farnesene | Step | Farnesene |
| Sample | Original | (g/L) | Volume (L) | (g) |
| Example | Whole cell broth | 45.5 | 17.5 | 797.0 |
| F1 | (WCB) | | | |
| Example | CCB | 239.5 | 2.5 | 598.8 |
| F2 | | | | |
| Example | Liquid/Liquid | 5.4 | 1.900 | 10.2 |
| F3 | Aqueous Phase | | | |
| Example | Crude farnesene | 798.7 | 0.600 | 479 |
| F4 | | | | |

TABLE 8
Concentration of farnesene in the liquid/liquid aqueous phase
obtained from samples with different extraction processes

| | | | | Liquid/ |
|---|---|---|---|---|
| | | | | Liquid |
| | | | | Aqueous |
| | | | | Phase |
| | | | Chemistry & | Titer |
| Sample | Run | Media | Concentration | (g/L) |
| Example | 4500 L | Chemically defined | pH/NaCl/ | 25 |
| F5 | CMO Run | medium | 0.5% L81 | |
| Example | 60000 L | Chemically defined | pH/NaCl/ | 30 |
| F6 | CMO Run | medium | 0.5% L81 | |
| Example | 60000 L | Chemically defined | pH/NaCl/ | 67 |
| F7 | CMO Run | medium | 0.5% L81 | |
| Example | 60000 L | Chemically defined | pH/NaCl/ | 52 |
| F8 | CMO Run | medium | 0.5% L81 | |
| Example | 60000 L | Chemically defined | pH/NaCl/ | 51 |
| F9 | CMO Run | medium | 0.5% L81 | |
| Example | 20 L | Chemically defined | 0.2% | 5 |
| F3 | Scaled | medium | TRITON ™ | |
| | Down | | X114/53° C. | |

### Example 8

### Effect of Surfactant Concentration on Farnesene Released from Cane Syrup Derived WCB at 40° C. and 50° C.

This example shows the effect of surfactant concentration on the amount of farnesene released from cane syrup derived WCB at incubation temperatures of 40° C. and 50° C. and demonstrates a similar effect of surfactant concentration on the amount of farnesene released from cane syrups derived WCB and CCB.

WCB without liquid/solid centrifugation was evaluated using Harvest broth from a 300 L fermentation utilizing cane syrup media.

Various concentrations of TRITON™ X114 ranged from about 0.01% to about 0.2% by v/v were added into the WCB, and then incubated for 30 minutes at 40° C. and 50° C. separately.

WCB incubated at 40° C. with TRITON™ X114 (0.01, 0.03, 0.07, 0.1 and 0.2% by v/v) were labeled as Examples G1-G5 respectively; whereas the WCB incubated at 50° C. with TRITON™ X114 (0.01, 0.03, 0.07, 0.1 and 0.2% by v/v) were labeled as Examples G6-G10 respectively.

A control experiment (Control G1) was done according to the procedure mentioned above except without the addition of surfactant. The oil release rate and oil recovery were measured by the Lumisizer at 4000 rpm (2300×g) at the incubation temperature for 22 minutes. Table 9 and 10 provide the oil recovery and oil release rate results of samples having different concentrations of TRITON™ X114 at 40° C. and 50° C.

The data suggest that the same absolute amount of TRITON™ X114 can be added to either WCB or CCB to provide similar yield improvement properties.

TABLE 9
The Oil release rates of Examples G1-G10 and Controls G1-G2

| | | Concen- | | |
|---|---|---|---|---|
| | | tration of | Incubation | Oil release |
| | Type of | Surfactant | Temperature | rate |
| Sample | Surfactant | (% by v/v) | (° C.) | (μm/sec) |
| Control G1 | — | — | 40 | 0.0429 |
| Example G1 | TRITON ™ X114 | 0.01 | 40 | 0.0892 |
| Example G2 | TRITON ™ X114 | 0.03 | 40 | 0.7925 |
| Example G3 | TRITON ™ X114 | 0.07 | 40 | 4.6976 |
| Example G4 | TRITON ™ X114 | 0.1 | 40 | 4.1887 |
| Example G5 | TRITON ™ X114 | 0.2 | 40 | 7.2393 |
| Control G2 | — | — | 50 | 0.0106 |
| Example G6 | TRITON ™ X114 | 0.01 | 50 | 0.0295 |
| Example G7 | TRITON ™ X114 | 0.03 | 50 | 0.8715 |
| Example G8 | TRITON ™ X114 | 0.07 | 50 | 4.4496 |
| Example G9 | TRITON ™ X114 | 0.1 | 50 | 2.8966 |
| Example | TRITON ™ X114 | 0.2 | 50 | 2.0327 |
| G10 | | | | |

TABLE 10
The Oil Recovery Results of Examples G1-G10 and Controls G1-G2

| | | | | | | % Clear Oil |
|---|---|---|---|---|---|---|
| | | Concentration | Incubation | | Clear Oil | In Emulsion |
| | Type of | of Surfactant | Temperature | Emulsion | Length | (which is equal to |
| Sample | Surfactant | (% by v/v) | (° C.) | Length (mm) | (mm) | oil recovery) |
| Control G1 | — | — | 40 | 2.12 | 0.45 | 21% |
| Example | TRITON ™ | 0.01 | 40 | 2.16 | 0.57 | 26% |
| G1 | X114 | | | | | |
| Example | TRITON ™ | 0.03 | 40 | 2.23 | 0.96 | 43% |
| G2 | X114 | | | | | |
| Example | TRITON ™ | 0.07 | 40 | 1.9 | 1.22 | 64% |
| G3 | X114 | | | | | |
| Example | TRITON ™ | 0.1 | 40 | 1.88 | 1.38 | 73% |
| G4 | X114 | | | | | |
| Example | TRITON ™ | 0.2 | 40 | 1.79 | 1.43 | 80% |
| G5 | X114 | | | | | |
| Control G2 | — | — | 50 | 2.22 | 0.58 | 26% |
| Example | TRITON ™ | 0.01 | 50 | 2.18 | 0.67 | 31% |
| G6 | X114 | | | | | |
| Example | TRITON ™ | 0.03 | 50 | 2.18 | 0.81 | 37% |
| G7 | X114 | | | | | |
| Example | TRITON ™ | 0.07 | 50 | 2.18 | 1.56 | 72% |
| G8 | X114 | | | | | |
| Example | TRITON ™ | 0.1 | 50 | 2.04 | 1.56 | 76% |
| G9 | X114 | | | | | |
| Example | TRITON ™ | 0.2 | 50 | 1.93 | 1.48 | 77% |
| G10 | X114 | | | | | |

There were more than two times increase in oil recovery of the samples (Example G3 and Example G8 respectively) with TRITON™ X114 (0.07% by v/v) at both incubation temperatures compared with that of the corresponding control experiments (Control G1 and Control G2 respectively).

The data suggest that the same absolute amount of TRITON™ X114 can be added to either WCB or CCB to provide similar yield improvement properties.

### Example 9

### Effect of Different Surfactants, i.e., TRITON™ X114 and TERGITOL™ L62, on Farnesene Released from Cane Syrup Derived WCB at 50° C. and 60° C.

This example shows the effect of different surfactants, including TRITON™ X114 and TERGITOL™ L62, on the amount of farnesene released from cane syrup derived WCB at incubation temperatures of 50° C. and 60° C. and demonstrates the difference in the effect of different surfactant on the amount of farnesene released from cane syrup derived WCB and CCB.

WCB (1 ml per tube) was aliquoted into the 1.5 ml microcentrifuge tubes. Different concentrations of TRITON™ X114 and TERGITOL™ L62 in an amount ranged from about 0.01% to about 0.1% were added into the tubes. The contents of each tube were then mixed at ambient temperature for 10 minutes by a vortex mixer. The tubes were then incubated at 50° C. and 60° C. for about 15 minutes. After incubation, the tubes were centrifuged at 4000 rpm (2300×g) for 22 minutes at the incubation temperatures.

The tubes with TRITON™ X114 (0.01, 0.03, 0.05, 0.07 and 0.1% by v/v) incubated at 50° C. were labeled as Examples H1-H5. The tubes with TERGITOL™ L62 (0.01, 0.03, 0.05, 0.07 and 0.1% by v/v) incubated at 50° C. were labeled as Examples H6-H10. The tubes with TRITON™ X114 (0.01, 0.03, 0.05, 0.07 and 0.1% by v/v) incubated at 60° C. were labeled as Examples H11-H15. The tubes with TERGITOL™ L62 (0.01, 0.03, 0.05, 0.07 and 0.1% by v/v) incubated at 60° C. were labeled as Examples H16-H20.

Control experiments (Controls H1-H2) were carried out according to the procedure mentioned above except without the addition of surfactant. The oil release rate and oil recovery of each sample were determined. Tables 11 and 12 provide the conditions and the oil release rate and oil recovery of the samples respectively.

The oil release rate of Controls H1-H2 and samples with TERGITOL™ L62 (Examples H6-H9, H16-H20) were found to be negative as shown in Table 11 which indicated a low oil breakout rate. The oil release rate of samples (Examples H1-H4, H12-H15) with TRITON™ X114 were found to be positive and the oil release rate generally increased with the concentration of TRITON™ X114 and the incubation temperature.

There was less discrepancy in the oil recovery between the samples with the same absolute amount of TRITON™ X114 and TERGITOL™ L62. The data suggest that although the oil release rates of the TRITON™ X114-containing samples were higher than that of the TERGITOL™ L62-containing samples, it did not necessarily translate into a much higher recovery of crude farnesene. This may be due to the fact that the oil release rate is an indication of centrifuge capacity for a given condition. The data suggest that the samples having TRITON™ X114 may allow a faster separation and higher throughput in the scaled process.

Example 9 demonstrates large performance differences between TRITON™ X-114 and TERGITOL™ L62 when applied to WCB. However, the performance differences between TRITON™ X114 and TERGITOL™ L62 when applied to CCB are minimal.

TABLE 11
Oil release rates of Examples H1-H20 and Controls H1-H2

| | | Concen- | | |
|---|---|---|---|---|
| | | tration of | Tem- | Oil release |
| | | Surfactant | perature | rates |
| Sample | Type of Surfactant | (% by v/v) | (° C.) | (μm/sec) |
| Control H1 | — | — | 50 | 0.0791 |
| Example H1 | TRITON ™ X114 | 0.01 | 50 | 0.057 |
| Example H2 | TRITON ™ X114 | 0.03 | 50 | 0.2335 |
| Example H3 | TRITON ™ X114 | 0.05 | 50 | 0.4165 |
| Example H4 | TRITON ™ X114 | 0.07 | 50 | 1.5299 |
| Example H5 | TRITON ™ X114 | 0.1 | 50 | 0.7911 |
| Example H6 | TERGITOL ™ L62 | 0.01 | 50 | −0.143 |
| Example H7 | TERGITOL ™ L62 | 0.03 | 50 | −0.0673 |
| Example H8 | TERGITOL ™ L62 | 0.05 | 50 | −0.1377 |
| Example H9 | TERGITOL ™ L62 | 0.07 | 50 | −0.0222 |
| Example H10 | TERGITOL ™ L62 | 0.1 | 50 | 0.0463 |
| Control H2 | — | — | 60 | −0.0672 |
| Example H11 | TRITON ™ X114 | 0.01 | 60 | 0.0231 |
| Example H12 | TRITON ™ X114 | 0.03 | 60 | 0.4487 |
| Example H13 | TRITON ™ X114 | 0.05 | 60 | 1.5305 |
| Example H14 | TRITON ™ X114 | 0.07 | 60 | 2.5505 |
| Example H15 | TRITON ™ X114 | 0.1 | 60 | 2.3575 |
| Example H16 | TERGITOL ™ L62 | 0.01 | 60 | −0.1838 |
| Example H17 | TERGITOL ™ L62 | 0.03 | 60 | −0.209 |
| Example H18 | TERGITOL ™ L62 | 0.05 | 60 | −0.2028 |
| Example H19 | TERGITOL ™ L62 | 0.07 | 60 | −0.1534 |
| Example H20 | TERGITOL ™ L62 | 0.1 | 60 | −0.0672 |

TABLE 12
The Oil Recovery Results of Examples H1-H20 and Controls H1-H2

| | | | | | | % Clear Oil |
|---|---|---|---|---|---|---|
| | | | | | | In Emulsion |
| | | Concentration | | Emulsion | Clear Oil | (which is |
| | Type of | of Surfactant | Temp. | Length | Length | equal to oil |
| Sample | Surfactant | (% by v/v) | (° C.) | (mm) | (mm) | recovery) |
| Control H1 | — | — | 50 | 1.59 | 0.57 | 36% |
| Example | TRITON ™ | 0.01 | 50 | N/A | N/A | N/A |
| H1 | X114 | | | | | |
| Example | TRITON ™ | 0.03 | 50 | 1.4 | 0.6 | 43% |
| H2 | X114 | | | | | |
| Example | TRITON ™ | 0.05 | 50 | 1.44 | 0.64 | 44% |
| H3 | X114 | | | | | |
| Example | TRITON ™ | 0.07 | 50 | 1.22 | 0.61 | 50% |
| H4 | X114 | | | | | |
| Example | TRITON ™ | 0.1 | 50 | 1.07 | 0.57 | 53% |
| H5 | X114 | | | | | |
| Example | TERGITOL ™ | 0.01 | 50 | N/A | N/A | N/A |
| H6 | L62 | | | | | |
| Example | TERGITOL ™ | 0.03 | 50 | 1.34 | 0.44 | 33% |
| H7 | L62 | | | | | |
| Example | TERGITOL ™ | 0.05 | 50 | 1.43 | 0.56 | 39% |
| H8 | L62 | | | | | |
| Example | TERGITOL ™ | 0.07 | 50 | 1.31 | 0.52 | 40% |
| H9 | L62 | | | | | |
| Example | TERGITOL ™ | 0.1 | 50 | 1.33 | 0.6 | 45% |
| H10 | L62 | | | | | |
| Control H2 | — | — | 60 | 1.75 | 0.71 | 41% |
| Example | TRITON ™ | 0.01 | 60 | N/A | N/A | N/A |
| H11 | X114 | | | | | |
| Example | TRITON ™ | 0.03 | 60 | 1.86 | 1 | 54% |
| H12 | X114 | | | | | |
| Example | TRITON ™ | 0.05 | 60 | 1.93 | 1.23 | 64% |
| H13 | X114 | | | | | |
| Example | TRITON ™ | 0.07 | 60 | 1.5 | 1.02 | 68% |
| H14 | X114 | | | | | |
| Example | TRITON ™ | 0.1 | 60 | 1.44 | 1.01 | 70% |
| H15 | X114 | | | | | |
| Example | TERGITOL ™ | 0.01 | 60 | 1.57 | 0.56 | 36% |
| H16 | L62 | | | | | |
| Example | TERGITOL ™ | 0.03 | 60 | 1.57 | 0.72 | 46% |
| H17 | L62 | | | | | |
| Example | TERGITOL ™ | 0.05 | 60 | 1.55 | 0.73 | 47% |
| H18 | L62 | | | | | |
| Example | TERGITOL ™ | 0.07 | 60 | 1.58 | 0.76 | 48% |
| H19 | L62 | | | | | |
| Example | TERGITOL ™ | 0.1 | 60 | 1.59 | 0.83 | 52% |
| H20 | L62 | | | | | |

Based on the above, the effects of TRITON™ X-114 at 0.25% v/v and TERGITOL™ L-62 at 0.25%, 0.5%, 0.75%, and 1.0% v/v were tested on CCB derived from very high polarity refined sucrose fermentations and subsequently heated to 60° C. and centrifuged to evaluate emulsion breakage. Under these conditions, all of the emulsions broke equally well except for the control samples (which were run under the same conditions except without surfactant). In another variation, a salt (NaCl varying from 5 g/L to 25 g/L) was added to the surfactant samples to see if the salt could further improve the amount of farnesene released from CCB. However, it was found that the salt in general had no additional impact on the amount of farnesene released from CCB.

Two other control experiments were conducted. In one control experiment, samples were treated as described in the previous paragraph where the surfactant was added except that the samples were not heated to a temperature above its respective PIT (or cloud point). In the second control experiment, surfactant was not added, but the samples were heated to a temperature above the PITs. On both control experiments, the respective samples had little or no farnesene release and were substantially similar to the samples which were neither treated with surfactant nor heated.

### Example 10

### Effect of Different Mixing Methods on the Oil Release Rate

The purpose of this example is to examine the possibility of reducing the time required for incubation by studying the effect of different mixing methods on the oil release rate.

The effect of mixing or power input on the amount of farnesene released from CCB was studied by utilizing different mixing equipment, including an ULTRA-TURRAX® disperser (commerically obtained from IKA®, Staufen, Germany), a stir bar at 1100 rpm and 600 rpm, a vortex mixer and a rotator mixer.

Firstly, two different lots of CCB were titrated with TERGITOL™ L62 to determine the quality of CCB. CCB was not demulsified fully by TERGITOL™ L62 but to significant degree of about 50% of CCB. The titration was carried out according to the titration procedure in Example 1. Based on the titration results, CCB (Lot. No.: PP051410F1_draw1) was used and TERGITOL™ L62 (0.1%) was added into each sample. All samples were mixed with the vortex mixer with maximum speed for 10 seconds at ambient temperature after the addition of TERGITOL™ L62. The samples were then mixed for certain time at ambient temperature with the following methods and conditions.

Vortex mixer (labeled as Example 13): CCB (5 ml) in a 15 ml conical bottom centrifuge tube was mixed at the beginning of each time of taking sample.

Rotating mixer (labeled as Example 14): CCB (5 ml) in a 15 ml conical bottom centrifuge tube was mounted to the tube rotator for mixing.

Stir bar (labeled as Examples 11 and 12): CCB (10 ml) was placed into a 25 ml scintillation vial and stirred with the stir bar at 1100 and 600 rpm respectively.

ULTRA-TURRAX® disperser (labeled as Example 15): CCB (20 ml) was placed into a 50 ml centrifuge tube and mixed continuously at 15000 rpm. The tube was placed into a water bath in order to remove heat generated in the process of mixing. Temperature of the sample was monitored during the mixing process to ensure the temperature of CCB was at ambient temperature.

Samples were taken from the tubes or vials and incubated in an oil bath at about 50° C. for 15 minutes.

After incubation at about 50° C., samples (400 μl) from the tubes were added into lumisizer microcentrifuge cells and analyzed by the Lumisizer. The samples in the Lumisizer were centrifuged at 4000 rpm (2300×g) at about 50° C. for 22 minutes.

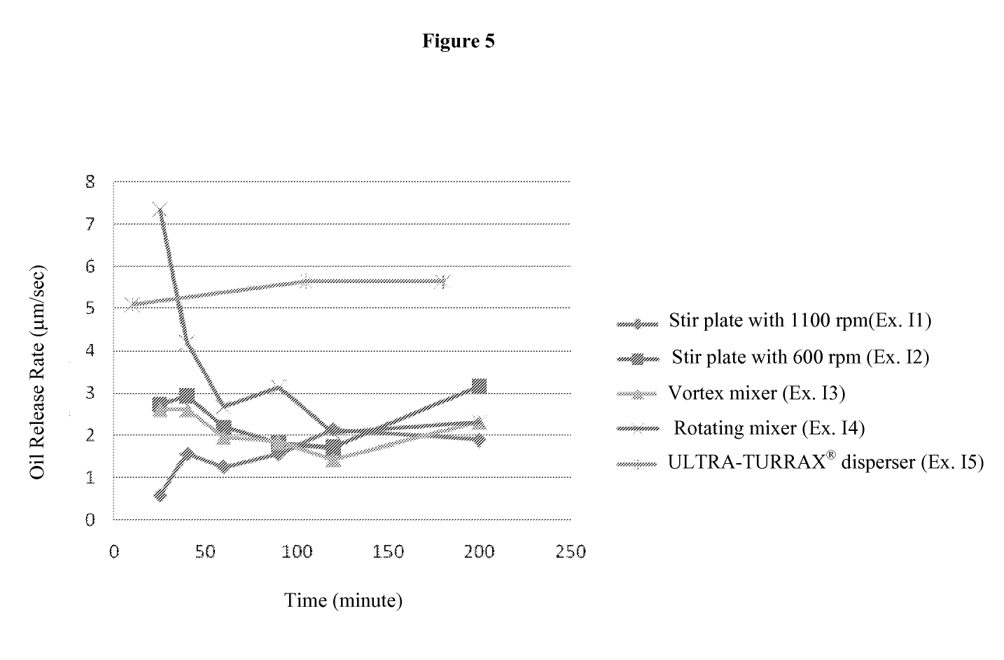

The oil release rates of the samples were determined and a plot of the oil release rate versus holding/mixing time with different mixing methods is shown in FIG. 5.

Referring to FIG. 5, Example I5 was found to have a high steady oil release rate starting as early as 10 minutes. The data in FIG. 5 indicate that mixing method can have significant effect on the oil release rate and thus the centrifuge capacity.

### Example 11

### Effect of Mixing Time on the Oil Release Rate of Samples Mixed with ULTRA-TURRAX® Disperser

Example 11 demonstrates the investigation on the minimum time for mixing samples with the ULTRA-TURRAX® disperser to achieve good mixing as indicated by the oil release rate.

The procedure for preparing Example J1 was as followed:

CCB (Lot No.: PP042310F1_draw3) (20 ml) was added into a 50 ml centrifuge tube and TERGITOL™ L62 (0.1% v/v) was added into the tube at ambient temperature. The mixture was mixed continuously at 15000 rpm for 15 minutes with the ULTRA-TURRAX® disperser. The tube was placed into a water bath in order to remove heat generated in the process of mixing. The temperature of the sample was monitored during the process to ensure the temperature of CCB was at ambient temperature. CCB was taken from the tube and incubated in the oil bath at 50° C. for 15 minutes.

Two control experiments (Controls J1-J2) were done. The first control experiment (Control J1) was done according to the procedure mentioned above except the content of the tube was mixed only by a vortex mixer at maximum speed for 5 seconds after the addition of TERGITOL™ L62 and without mixing with the ULTRA-TURRAX® disperser. The second control experiment (Control J2) was done according to the procedure mentioned above except without the addition of TERGITOL™ L62 and without mixing with the ULTRA-TURRAX® disperser.

At different time intervals, samples (400 μl) from the tubes was added into lumisizer microcentrifuge cells and analyzed by the Lumisizer. The samples in the Lumisizer were centrifuged at 4000 rpm (2300×g) at 50° C. for 22 minutes.

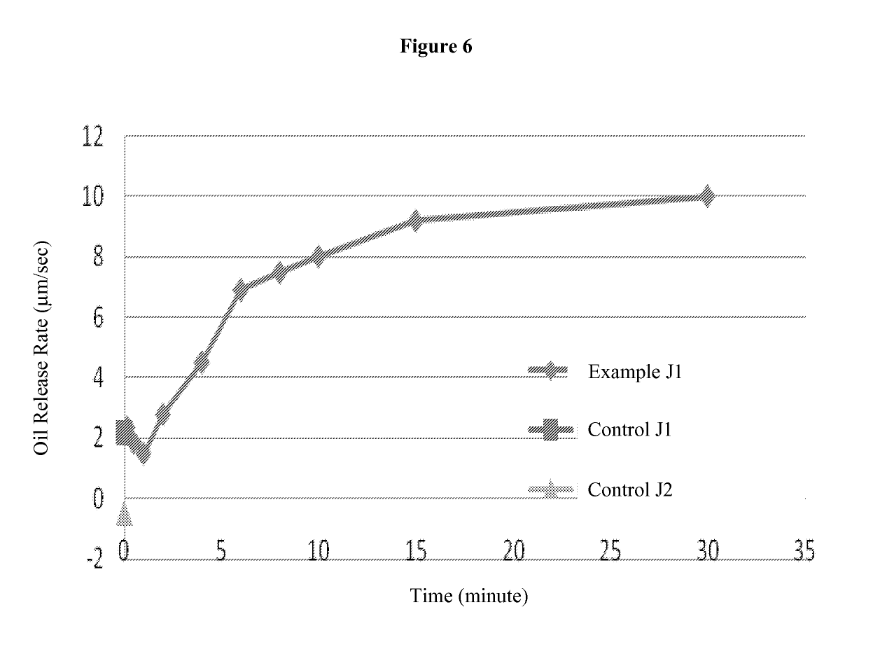

The oil release rates of the samples were determined and a plot of the oil release rate versus the mixing time with the ULTRA-TURRAX® disperser is shown in FIG. 6.

The data suggests that there is a significant increase in oil release rate of Example J1 in the first 10 minutes compared with the oil release rate obtained from Control J1.

### Example 12

### Effect of Different Mixing Methods and the Concentration of TERGITOL™ L62 on the Oil Recovery and Oil Release Rate

This example shows the effect of different mixing methods and the concentration of TERGITOL™ L62 on the oil recovery and oil release rate.

Example 12 evaluated the amount of TERGITOL™ L62 required to give optimal farnesene release under “low mix” and “high mix” regimes. The effectiveness of demulsification of samples having different concentrations of TERGITOL™ L62 was studied using two mixing equipment, the stir bar and ULTRA-TURRAX® disperser. The procedure of Example 12 was as followed:

Stir bar (labeled as Example K1): CCB (Lot No.: PP052110F2_draw1) (2 ml per vial) was aliqouted into 4 ml scintillation vials. Then TERGITOL™ L62 in different amounts ranged from 0 to 0.5% by v/v was added into the vials. After the addition of the TERGITOL™ L62, each sample was mixed by a vortex mixer for 5 seconds at maximum speed at ambient temperature. The contents in each vial were then mixed periodically at maximum speed with vortex mixer for 15 minutes at ambient temperature.

ULTRA-TURRAX® disperser (labeled as Example K2): CCB (Lot No.: PP052110F2_draw1) (2 ml per tube) was aliqouted into each 15 ml conical bottom centrifuge tubes. Then TERGITOL™ L62 in different amounts ranged from 0 to 0.5% by v/v was added into the tubes. After the addition of the TERGITOL™ L62, each sample was mixed by a vortex mixer for 5 seconds at maximum speed at ambient temperature. Then the contents in each tube were mixed with ULTRA-TURRAX® disperser at 15000 rpm for 15 minutes at ambient temperature. The tube was placed into a water bath in order to remove heat generated in the process of mixing.

CCB was taken from the vials and the tubes and incubated in an oil bath at about 60° C. for 15 minutes.

Samples (400 μl) from the tubes were added into lumisizer microcentrifuge cells and analyzed by the Lumisizer. The samples in the Lumisizer were centrifuged at 4000 rpm (2300×g) at about 60° C. for 22 minutes.

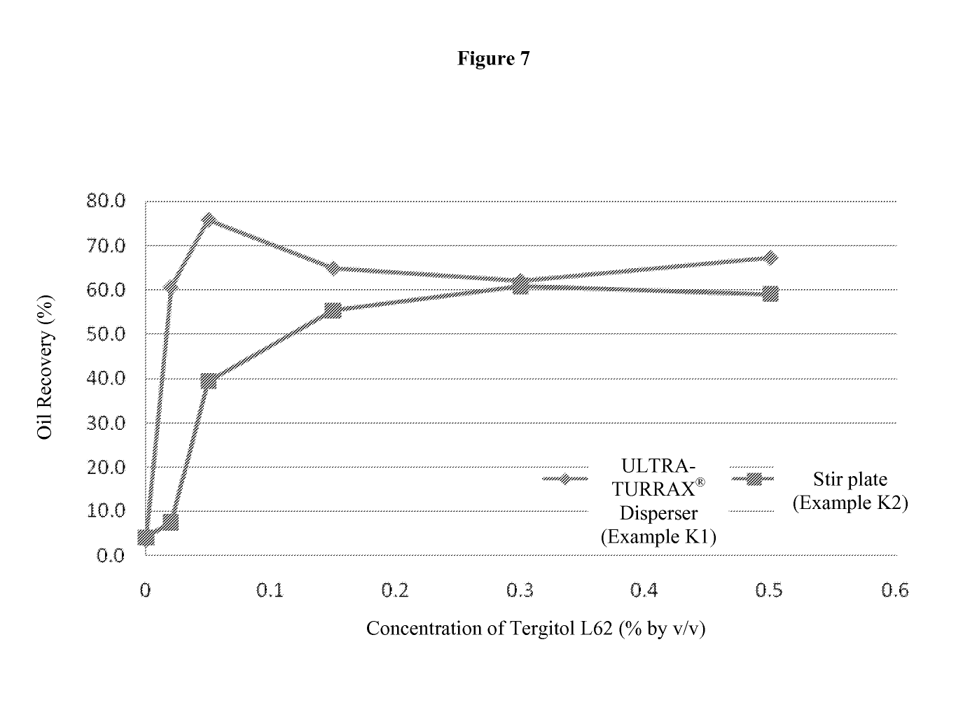

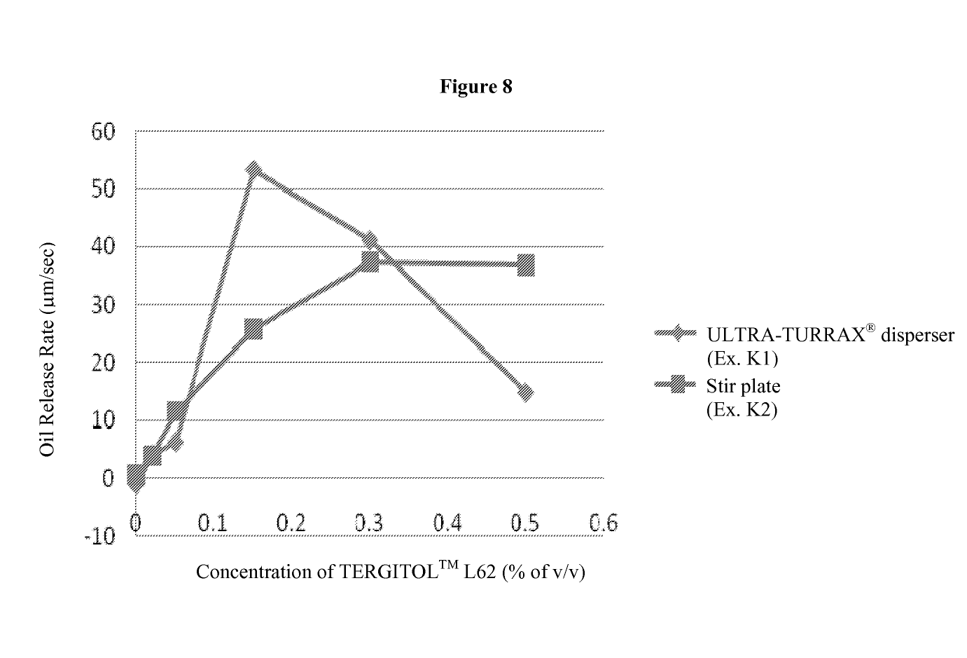

The oil recovery and oil release rate of each sample was determined and plots of the oil recovery and oil release rate versus the concentration of TERGITOL™ L62 are shown in FIGS. 7 and 8 respectively.

Referring to FIG. 7, the oil recovery of Example K1 increased sharply with the concentration of TERGITOL™ L62. On the other hand, Example K2 had a more gradual response in oil release rate. More importantly, the oil recovery of Example K1 was significantly higher than that of Example K2 when the concentrations of TERGITOL™ L62 were lower than 0.1% by v/v such as 0.02% and 0.05% by v/v.

On the other hand, there might be a critical concentration range for TERGITOL™ L62 to achieve a maximum oil release rate when the ULTRA-TURRAX® disperser was used for mixing. The plot shown in FIG. 8 shows that the critical concentration range of TERGITOL™ L62 was from 0.1 to 0.2% by v/v. This suggests that the concentration of TERGITOL™ L62 may need to be optimized to achieve desired oil recovery and oil release rate.

### Example 13

### Displacement of Protein after the Addition of Surfactant

The data in this example show that proteins are a main bio-emulsifier present in the farnesene emulsion. Protein may be displaced after the addition of TERGITOL™ L62, which is consistent with the transformation from a bio-emulsion to a chemical emulsion.

The aqueous phase protein content of a sample by bicinchoninic acid protein assay (BCA) (Bovine Serum Albumin (BSA) standard curve) was found to be 0.95 g/L before TERGITOL™ 62 addition, and 1.84 g/L after TERGITOL™ L62 addition.

Other data (not shown) demonstrated that protease treatment reduced the size of the emulsion, further supporting the hypothesis that proteins stabilize the farnesene emulsion.

### Example 14

### Comparison of Process Yield Between Previous Liquid Separation Process and the New Liquid Separation Process from Cane Syrup CCB

The process yield of three previous liquid separation processes and an embodiment of the inventive liquid separation process from cane syrup CCB are shown in Tables 13 and 14 respectively.

TABLE 13
Process yields of previous liquid separation
processes from cane syrup CCB

| | Liquid Yield | |
|---|---|---|
| Run | (%) | Chemistry |
| 073009C2 (Y1551, Un-clarified syrup) | 76 | pH 9.5/0.5% L- |
| Campinas (Y1551, Un-clarified syrup) | 78-92 | 81/0.65M NaCl |
| 082809C1 (Y2450, low solids syrup) | 77 | |

TABLE 14
Process yield of an embodiment of the inventive liquid
separation process from cane syrup CCB
Tropicalized DSP Yield on 2 Jun. 2010 (N = 4)
Liquid Yield (%)
98.5 ± 0.2

### Example 15

### Large Scale Farnesene Separation Process

A continuous disk stack nozzle centrifuge (Alfa Laval DX203 B-34) was used to separate cells from the fermentation broth. The liquid/solid centrifuge was fed directly from the fermentor, or the fermentation broth or fermentation harvest broth was transferred to a harvest tank or hold tank. The tank used to feed the centrifuge was mixed and temperature was controlled at about 30° C.-35° C. In the batch process, about 85% of the volumetric flow, which contained cells and one or more liquids, exited from the nozzles of the centrifuge, while about 15% of the volumetric flow was captured as CCB. The heat exchanger/centrifuge feed flow rate was about 14,000 L/hr. This process substantially reduced the volume which needed to be separated in the three-phase separation step. The farnesene at this stage was presented either as a clear product, or in an emulsified state with water and cells.

The harvest cell broth was held in the harvest tank for about 24-48 hours at about 4° C. to about 8° C. before processing through the liquid/solid centrifuge. The harvest was warmed to about 30° C. before processing through the liquid/solid centrifuge.

The Liquid/Solid centrifugation product, i.e., CCB, was stored at about 4° C. to about 8° C. up to about 72 hours before the next step. CCB was warmed to ambient temperature before the next step.

The transfer/feed lines and the tank seals were selected to be chemically or physically compatible with the farnesene product. For example, VITON® lines and seals were selected whereas EPDM lines and seals were not.

CCB was treated to reduce the level of emulsification prior to the liquid/liquid separation. The treatment was accomplished by two steps: (a) the addition of TRITON™ X114 (0.25% by v/v) to CCB, and (b) in-line heating of the mixture of CCB and TRITON™ X114. After the addition of the TRITON™ X114 to CCB, the mixture was mixed for about 1.5-2 hours at ambient temperature (up to about 30° C.) before the next step. The mixture was stored for up to about 3 days at about 4° C. to 8° C. before liquid/liquid separation with no adverse effects on product recovery.

A continuous, three-phase, disk-stack centrifuge was used to separate the clear farnesene phase from the heavy aqueous phase and solids. Prior to feeding the three-phase centrifuge, the mixture of CCB and TRITON™ X114 was de-emulsified by heating the mixture in-line. The mixture was fed through a heat exchanger where the mixture was heated to about 60° C. for about 30 seconds. After passing through the heat exchanger, the product was fed into the centrifuge with a feed flow rate of 2,000-4,000 L/hour. The light and heavy phases exited through respective outlets into bowls. Solids gradually accumulated in the bowl and was discharged periodically to maintain the separation efficiency.

The residual solids in the crude farnesene phase were removed as a last step using either liquid/solid centrifugation or filtration. After the polishing step, an anti-oxidant (100 ppm w/w) (e.g. tert-butyl catechol) was added to the crude farnesene to stabilize the product for storage and shipment. The yield of the crude farnesene by this process was about 70-90% based on measuring the content of farnesene with GC-FID analysis. The purity of the crude farnesene was about 95%.

The examples set forth above are provided to give those of ordinary skill in the art with a complete disclosure and description of how to make and use the claimed embodiments and are not intended to limit the scope of what is disclosed herein. Modifications that are obvious to persons of skill in the art are intended to be within the scope of the following claims. All publications, patents and patent applications cited in this specification are incorporated herein by reference as if each such publication, patent or patent application were specifically and individually indicated to be incorporated herein by reference.

What is claimed is:

1. A method comprising:
(a) providing a composition comprising a surfactant, host cells, an aqueous medium and a bio-organic compound produced by the host cells, wherein the solubility of the surfactant in the aqueous medium decreases with increasing temperature and wherein the temperature of the composition is at least about 1° C. below a phase inversion temperature of the composition;
(b) raising the temperature of the composition to at least about 1° C. above the phase inversion temperature; and
(c) performing a liquid/liquid separation of the composition to provide a crude bio-organic composition.

2. The method of claim 1 further comprising a step of reducing the volume of the composition before step (b), wherein substantially all of the bio-organic compound remains in the composition.

3. The method of claim 2, wherein the volume of the composition is reduced by about 75% or more.

4. The method of claim 1, wherein the surfactant comprises a non-ionic surfactant.

5. The method of claim 4, wherein the non-ionic surfactant is a polyether polyol, a polyoxyethylene C₈₋₂₀-alkyl ether, a polyoxyethylene C₈₋₂₀-alkylaryl ether, a polyoxyethylene C₈₋₂₀-alkyl amine, a polyoxyethylene C₈₋₂₀-alkenyl ether, a polyoxyethylene C₈₋₂₀-alkenyl amine, a polyethylene glycol alkyl ether or a combination thereof.

6. The method of claim 4, wherein the non-ionic surfactant is a polyether polyol, polyoxyethylene nonyl phenyl ether, polyoxyethylene dedecyl phenyl ether or a combination thereof.

7. The method of claim 1, wherein the temperature in step (a) is at least about 5° C. or at least about 10° C. below the phase inversion temperature.

8. The method of claim 1, wherein the temperature in step (b) is raised to at least about 5° C., or at least about 10° C., or at least about 15° C. above the phase inversion temperature.

9. The method of claim 1, wherein the bio-organic compound is a hydrocarbon, or an isoprenoid, or a farnesene.

10. The method of claim 9, wherein the farnesene is an α-farnesene, β-farnesene or a combination thereof.

11. The method of claim 1, wherein the host cells are bacteria, fungi, algae or a combination thereof.

12. The method of claim 1, wherein the host cells are selected from the genera Escherichia, Bacillus, Lactobacillus, Kluyveromyces, Pichia, Saccharomyces, Yarrowia, S. cerevisiae, Chlorella minutissima, Chlorella emersonii, Chloerella sorkiniana, Chlorella ellipsoidea, Chlorella sp. Chlorella protothecoides and combinations thereof.

13. The method of claim 1, wherein the method further comprises purifying the crude bio-organic composition to yield a purified bio-organic composition.

14. The method of claim 13, wherein the purification of the crude bio-organic composition is by flash distillation.

15. The method of claim 13 further comprising treating the purified bio-organic composition with an antioxidant.

16. The method of claim 15, wherein the antioxidant is a phenolic antioxidant.

17. A composition comprising a surfactant, host cells, an aqueous medium and a bio-organic compound produced by the host cells, wherein the solubility of the surfactant in the aqueous medium decreases with increasing temperature and wherein the temperature of the composition is at least about 1° C. above a phase inversion temperature of the composition.

18. The composition of claim 17, wherein the surfactant comprises a non-ionic surfactant.

19. The composition of claim 18, wherein the non-ionic surfactant is a polyether polyol, a polyoxyethylene C₈₋₂₀-alkyl ether, a polyoxyethylene C₈₋₂₀-alkylaryl ether, a polyoxyethylene C₈₋₂₀-alkyl amine, a polyoxyethylene C₈₋₂₀-alkenyl ether, a polyoxyethylene C₈₋₂₀-alkenyl amine, a polyethylene glycol alkyl ether or a combination thereof.

20. The composition of claim 18, wherein the non-ionic surfactant is a polyether polyol, polyoxyethylene nonyl phenyl ether, polyoxyethylene dedecyl phenyl ether or a combination thereof.

21. The composition of claim 17, wherein the temperature of the composition is at least about 5° C., or at least about 10° C., or at least about 15° C. above the phase inversion temperature.

22. The composition of claim 17, wherein the bio-organic compound is a hydrocarbon, or an isoprenoid, or a farnesene.

23. The composition of claim 22, wherein the farnesene is an α-farnesene, β-farnesene or a combination thereof.

24. The composition of claim 17, wherein the host cells are bacteria, fungi, algae or a combination thereof.

25. The composition of claim 17, wherein the host cells are selected from the genera Escherichia, Bacillus, Lactobacillus, Kluyveromyces, Pichia, Saccharomyces, Yarrowia, S. cerevisiae, Chlorella minutissima, Chlorella emersonii, Chloerella sorkiniana, Chlorella ellipsoidea, Chlorella sp., Chlorella protothecoides and combinations thereof.

26. The composition of claim 17, wherein the composition is an emulsion.

27. A method comprising:
(a) providing an oil-in-water emulsion comprising a surfactant, host cells, an aqueous medium and a bio-organic compound produced by the host cells, wherein the solubility of the surfactant in the aqueous medium decreases with increasing temperature;
(b) converting the oil-in-water emulsion to a water-in-oil emulsion; and
(c) performing a liquid/liquid separation of the water-in-oil emulsion to provide a crude bio-organic composition.

28. The method of claim 27 further comprising a step of reducing the volume of the oil-in-water emulsion before step (b), wherein substantially all of the bio-organic compound remains in the oil-in-water emulsion.

29. The method of claim 27, wherein the volume of the oil-in-water emulsion is reduced by about 75% or more.

30. The method of claim 27, wherein the surfactant comprises a non-ionic surfactant.

31. The method of claim 30, wherein the non-ionic surfactant is a polyether polyol, a polyoxyethylene C₈₋₂₀-alkyl ether, a polyoxyethylene C₈₋₂₀-alkylaryl ether, a polyoxyethylene C₈₋₂₀-alkyl amine, a polyoxyethylene C₈₋₂₀-alkenyl ether, a polyoxyethylene C₈₋₂₀-alkenyl amine, a polyethylene glycol alkyl ether or a combination thereof.

32. The method of claim 30, wherein the non-ionic surfactant is a polyether polyol, polyoxyethylene nonyl phenyl ether, polyoxyethylene dedecyl phenyl ether or a combination thereof.

33. The method of claim 27, wherein the bio-organic compound is a hydrocarbon, or an isoprenoid, or a farnesene.

34. The method of claim 33, wherein the farnesene is an α-farnesene, β-farnesene or a combination thereof.

35. The method of claim 27, wherein the host cells are bacteria, fungi, algae or a combination thereof.

36. The method of claim 27, wherein the host cells are selected from the genera Escherichia, Bacillus, Lactobacillus, Kluyveromyces, Pichia, Saccharomyces, Yarrowia, S. cerevisiae, Chlorella minutissima, Chlorella emersonii, Chloerella sorkiniana, Chlorella ellipsoidea, Chlorella sp., Chlorella protothecoides and combinations thereof.

37. The method of claim 27, wherein the method further comprises purifying the crude bio-organic composition to yield a purified bio-organic composition.

38. The method of claim 37, wherein the purification of the crude bio-organic composition is by flash distillation.

39. The method of claim 38 further comprising treating the purified bio-organic composition with an antioxidant.

40. The method of claim 39, wherein the antioxidant is a phenolic antioxidant.

41. The method of claim 2, wherein the surfactant comprises a non-ionic surfactant.

42. The method of claim 41, wherein the non-ionic surfactant is a polyether polyol, a polyoxyethylene C₈₋₂₀-alkyl ether, a polyoxyethylene C₈₋₂₀-alkylaryl ether, a polyoxyethylene C₈₋₂₀-alkyl amine, a polyoxyethylene C₈₋₂₀-alkenyl ether, a polyoxyethylene C₈₋₂₀-alkenyl amine, a polyethylene glycol alkyl ether or a combination thereof.

43. The composition of claim 26, wherein the surfactant comprises a non-ionic surfactant.

44. The composition of claim 43, wherein the non-ionic surfactant is a polyether polyol, a polyoxyethylene C₈₋₂₀-alkyl ether, a polyoxyethylene C₈₋₂₀-alkylaryl ether, a polyoxyethylene C₈₋₂₀-alkyl amine, a polyoxyethylene C₈₋₂₀-alkenyl ether, a polyoxyethylene C₈₋₂₀-alkenyl amine, a polyethylene glycol alkyl ether or a combination thereof.

45. The method of claim 28, wherein the surfactant comprises a non-ionic surfactant.

46. The method of claim 45, wherein the non-ionic surfactant is a polyether polyol, a polyoxyethylene C₈₋₂₀-alkyl ether, a polyoxyethylene C₈₋₂₀-alkylaryl ether, a polyoxyethylene C₈₋₂₀-alkyl amine, a polyoxyethylene C₈₋₂₀-alkenyl ether, a polyoxyethylene C₈₋₂₀-alkenyl amine, a polyethylene glycol alkyl ether or a combination thereof.
